(19) United States
(12) Patent Application Publication
(10) Pub. No.: US 2021/0134431 A1
(43) Pub. Date: May 6, 2021

(54) MEDICAL FLUID DELIVERY SYSTEM INCLUDING ANALYTICS FOR MANAGING PATIENT ENGAGEMENT AND TREATMENT COMPLIANCE

(71) Applicants: BAXTER INTERNATIONAL INC., Deerfield, IL (US); BAXTER HEALTHCARE SA, Glattpark (Opfikon) (CH)

(72) Inventors: Christy Elizabeth Garcia, Gurnee, IL (US); Timothy Louis Kudelka, Lindenhurst, IL (US); Jonathan Alan Handler, Northbrook, IL (US); Ion Janos Kelemen, Eching (DE); Mark Anthony Penny, Thetford (GB); Angelo A. Sarto, Franksville, WI (US); Andrew Thomas Gebhardt, Lake Zurich, IL (US); Ashkan Khorzad, Barrington, IL (US); Richard Scott Tessell, Highwood, IL (US)

(21) Appl. No.: 17/090,468

(22) Filed: Nov. 5, 2020

Related U.S. Application Data
(60) Provisional application No. 62/930,889, filed on Nov. 5, 2019.

(51) Int. Cl. G16H 20/40; G16H 10/60; G16H 40/20; G16H 40/67; G16H 50/20; G16H 70/20; G16H 50/30; G16H 50/70; A61B 5/00; A61M 1/28

(52) U.S. Cl. CPC G16H 20/40; A61M 1/3403; G16H 40/20; G16H 40/67; G16H 50/20; G16H 70/20; G16H 50/30; G16H 50/70; A61B 5/4833; A61B 5/742; A61B 5/746; A61M 1/285; A61M 1/282; A61M 2205/52; A61M 2205/50; A61M 2205/502; A61M 2205/3584; A61M 2205/702; A61M 2205/18; A61M 2205/3389; G16H 10/60

(57) ABSTRACT

A medical fluid delivery system including analytics for managing patient engagement and treatment compliance is disclosed. In an example, an interface device receives treatment data from a dialysis machine for a patient undergoing dialysis. An analytics processor determines dialysis treatment parameter values by comparing the treatment data to a record of a prescribed therapy or program. The dialysis treatment parameter values may include a lost treatment time parameter value, a lost dwell time parameter value, and/or a completed treatment days parameter value. The analytics processor causes the dialysis treatment parameter values to be displayed within a user interface on a clinician device. The dialysis treatment parameter values provide an indication as to how well the patient is complying with the prescribed therapy or program, and may be used by a clinician to help a patient improve compliance if needed.

### PRIORITY CLAIM

This application claims priority to and the benefit of U.S. Provisional Patent Application Ser. No. 62/930,889, filed on Nov. 5, 2019, the entire disclosure of which is hereby incorporated by reference.

### BACKGROUND

Engaging a patient outside of a medical environment for an extended period is currently a virtually impossible task. Similar to beginning a gym membership or buying a treadmill, many patients typically begin strong. Initially, a patient is readily engaged with a self-administered medical treatment (e.g., a medical fluid delivery treatment). Oftentimes, these medical treatments are conducted in a patient's home and/or in a clinic. For medical fluid delivery treatments, a patient has to connect themselves to a medical fluid delivery machine (or containers that contain a renal failure treatment fluid) to cleanse their blood to counter a build-up of toxins. Part of the treatment may include administrative tasks that the patient has to perform such as weighing themselves, taking their blood pressure, and/or recording information related to their treatment. The information recorded by the patient is oftentimes reviewed by clinicians to ensure the treatment is progressing as prescribed. Clinicians also review the recorded data to determine whether an adjustment to the treatment is needed.

Overtime, patients become less enthusiastic as the repetition of the treatments lose their novelty and become just another daily obligation. As one can imagine, a patient would rather engage in more exciting, relaxing, or stimulating activities compared to a self-administered medical treatment or traveling to a clinic multiple times a week for the same treatment. While many patients continue the treatments, they sometimes begin to omit performing the additional tasks that go along with the treatments. Omitting the additional tasks and becoming less enthused with treatments has the potential to create gaps in clinical oversight for an ongoing treatment. As patients become further disengaged from the treatments, they may begin to skip treatments, perform the treatments for only a portion of a prescribed time, or forgo them altogether, risking their health in the process.

### SUMMARY

A medical fluid data transfer system for determining and/or predicting patient adherence is disclosed herein. The medical fluid data transfer system is configured to improve a patient's treatment compliance by tracking how the patient uses or otherwise interacts with a medical fluid delivery machine, such as an automated peritoneal dialysis (“APD”) machine. In some embodiments, the medical fluid data transfer system analyzes data from a medical fluid delivery machine to determine a patient's adherence to one or more prescribed therapies or programs. If patient adherence has fallen below a specified threshold or is trending to fall below a threshold, the medical fluid data transfer system may operate one or more evidence-based algorithmic models. As disclosed herein, the models are configured to provide recommendations to clinicians regarding how patient adherence to one or more prescribed therapies or programs can be improved by addressing potential issues the patient may be experiencing.

In some embodiments, the medical fluid data transfer system may alternatively or additionally include one or more artificial intelligence (“AI”) patient predictive models that are configured to identify patients at risk of stopping their treatments or falling below a specified adherence threshold. The one or more AI patient predictive models are configured to determine a concern score, which is indicative of a probability that a given patient may end a treatment or fall below a desired adherence threshold. The AI patient predictive models determine the concern score by taking into account treatment data from a medical fluid delivery machine and readily available patient information as it relates to a prescribed therapy. As such, the AI patient predictive models are configured to accurately determine a patient's risk using readily available data without having to access third-party data or other medical data that is stored in a patient's medical record. In addition to providing a concern score, the example AI patient predictive models described herein are configured to provide visibility or insight regarding how the concern score is calculated. For instance, the AI patient predictive models may provide a number of significant reasons or attributes that contributed to the concern score. In some embodiments, the medical fluid data transfer system may generate adherence recommendations for a clinician based on the significant attributes identified by the AI patient predictive model.

In addition to analyzing patient adherence to one or more prescribed therapies or programs performed by a medical fluid delivery machine, the medical fluid data transfer system disclosed herein may also track alarm fatigue and/or determine if a patient's catheter is experiencing an issue. In an example, the medical fluid data transfer system may track a number and types of alarms generated by a medical fluid delivery machine for a specific patient. The medical fluid delivery machine may identify an issue related to the one or more prescribed therapies or programs based on the alarms. The medical fluid delivery machine may then use one or more evidence based and/or AI models to provide recommendations for avoiding alarms generated for the patient or a related clinician. The medical fluid data transfer system may also enable a clinician and/or the patient to silence some alarms or address alarms in an attempt to limit or avoid patient alarm fatigue.

In another example, the medical fluid data transfer system is configured to receive fill and drain data from a medical fluid delivery machine to determine catheter status. The medical fluid delivery machine may determine that a patient's catheter placement is incorrect or a leak is present if a fill or drain time for one or more prescribed therapies or programs is less than a specified threshold. Further, the medical fluid data transfer system may determine a catheter is partially blocked or has an accumulation of fibrin strands if a fill or drain time for one or more prescribed therapies or programs is greater than a specified threshold.

The example medical fluid data transfer system accordingly determines a holistic picture of a patient's treatment results, which is provided as adherence information. The medical fluid data transfer system may use the adherence information for providing recommendations and/or guidance to improve patient adherence to the one or more prescribed therapies and/or programs. Further, the medical fluid data transfer system may predict patients that are at risk for decreasing or stopping their treatments altogether. The medical fluid data transfer system disclosed herein accordingly improves patient adherence to one or more prescribed therapies or programs by tracking and encouraging their interaction and use of one or more medical fluid delivery machines. In other words, the medical fluid data transfer system provides transparency into a patient's treatment, which enables clinicians to intervene early when treatment data is indicative that an adherence problem is developing or is in the early stages of developing. This transparency accordingly enables clinicians to intervene before a patient's medical condition worsens.

The medical fluid data transfer system and methodology of the present disclosure is applicable, for example, to fluid delivery for plasmapherisis, hemodialysis (“HD”), hemofiltration (“HF”) hemodiafiltration (“HDF”), and continuous renal replacement therapy (“CRRT”) treatments. The medical fluid data transfer system described herein is also applicable to peritoneal dialysis (“PD”), intravenous drug delivery, and nutritional fluid delivery. These modalities may be referred to herein collectively or generally individually as a medical fluid delivery or treatment.

The above modalities may be provided by a medical fluid delivery machine that houses components needed to deliver medical fluid, such as one or more pumps, valves, heaters (if needed), online medical fluid generation equipment (if needed), sensors, such as any one, or more, or all of pressure sensors, conductivity sensors, temperature sensors, air detectors, blood leak detectors, and the like, user interfaces, and control units, which may employ one or more processors and memory to control the above-described equipment. The medical fluid delivery machine may also include one or more filters, such as a dialyzer or hemofilter for cleansing blood and/or an ultrafilter for purifying water, dialysis fluid, or other fluid.

The medical fluid delivery machine and the medical fluid data transfer system and methodology described herein may be used with home-based machines. For example, the systems may be used with home HD, HF or HDF machines, which are operated at the patient's convenience. One such home system is described in U.S. Pat. No. 8,029,454 (“the '454 Patent”), issued Oct. 4, 2011, entitled “High Convection Home Hemodialysis/Hemofiltration And Sorbent System”, filed Nov. 4, 2004, assigned to the assignees of the present application. Other such home systems are described in U.S. Pat. No. 8,393,690 (“the '690 Patent”), issued Mar. 12, 2013, entitled “Enclosure for a Portable Hemodialysis System”, filed Aug. 27, 2008. The entire contents of each of the above references are incorporated herein by reference and relied upon.

As described in detail below, the medical fluid data transfer system and methodology of the present disclosure may operate within an encompassing platform or system that may include many machines comprising many different types of devices, patients, clinicians, doctors, service personnel, electronic medical records (“EMR”) databases, a website, a resource planning system handling data generated via the patient and clinician communications, and business intelligence. The medical fluid data transfer system and methodology of the present disclosure operates seamlessly within the overall system and without contravening its rules and protocols.

In light of the disclosure herein and without limiting the disclosure in any way, in a first aspect of the present disclosure, which may be combined with any other aspect listed herein unless specified otherwise, a system for managing patient adherence to a prescribed therapy or program that is administered by an automated peritoneal dialysis (“APD”) machine includes a memory device storing a record of the prescribed therapy or program for a patient including a schedule of days a treatment is to be provided, a prescribed treatment duration, and an estimated prescribed treatment dwell time. The memory device also stores treatment data generated by the APD machine. The treatment data is indicative of a treatment duration, a fluid dwell time, and date for each dialysis treatment performed by the APD machine according to the prescribed treatment or program. The system also includes an interface device communicatively coupled to the APD machine via a network. The interface is configured to receive the treatment data from the APD machine. The system further includes an analytics processor communicatively coupled to the interface device and the memory device. The analytics processor is configured to store the treatment data received by the interface device to the memory device, determine a lost treatment time parameter value as a difference or ratio between the prescribed treatment duration and the treatment durations of the dialysis treatments, determine a lost dwell time parameter value as a difference or ratio between the estimated prescribed treatment dwell time and the fluid dwell time of the dialysis treatments, and determine a completed treatment days parameter value as a difference or ratio between the schedule of days the treatment is to be provided and the date of the dialysis treatments. The analytics processor is also configured to cause the lost treatment time parameter value, the lost dwell time parameter value, and the completed treatment days parameter value to be displayed within a user interface on a clinician device.

In accordance with a second aspect of the present disclosure, which may be used in combination with any other aspect listed herein unless stated otherwise, the memory device includes a data structure that relates medical fluid delivery recommendations to at least one of a range of lost treatment time parameter values, a range of lost dwell time parameter values, or a range of completed treatment days parameter values.

In accordance with a third aspect of the present disclosure, which may be used in combination with any other aspect listed herein unless stated otherwise, the system further includes a guidance processor communicatively coupled to the memory device. The guidance processor is configured to compare at least one of the lost treatment time parameter value, the lost dwell time parameter value, or the completed treatment days parameter value for the patient to the respective at least one of the range of lost treatment time parameter values, the range of lost dwell time parameter values, or the range of completed treatment days parameter values, select at least one recommendation based on the comparison, and cause the at least one recommendation to be displayed within the user interface on the clinician device.

In accordance with a fourth aspect of the present disclosure, which may be used in combination with any other aspect listed herein unless stated otherwise, the analytics processor is configured to store the lost treatment time parameter value, the lost dwell time parameter value, and the completed treatment days parameter value to the memory device in association with previous lost treatment time parameter values, previous lost dwell time parameter values, and previous completed treatment days parameter values from previous treatments of the patient, create a first graph using the current and previous lost treatment time parameter values to show actual treatment times for the patient compared to the prescribed treatment duration of the treatments, and cause the first graph to be displayed in a first user interface of the clinician device.

In accordance with a fifth aspect of the present disclosure, which may be used in combination with any other aspect listed herein unless stated otherwise, the analytics processor is configured to create a second graph using the current and previous lost dwell time parameter values to show actual dwell times for the patient compared to the estimated prescribed treatment dwell time, and cause the second graph to be displayed in a second user interface of the clinician device.

In accordance with a sixth aspect of the present disclosure, which may be used in combination with any other aspect listed herein unless stated otherwise, the record of the prescribed therapy or program for the patient includes at least one of a fill threshold or a drain threshold, and the treatment data includes at least one of a fluid fill time or a fluid drain time for the treatment. The analytics processor is configured to generate an alert if the at least one of the fluid fill time, a weekly average of fluid fill times, the fluid drain time, or a weekly average of fluid drain times exceeds the respective at least one of the fill threshold or the drain threshold.

In accordance with a seventh aspect of the present disclosure, which may be used in combination with any other aspect listed herein unless stated otherwise, the alert is indicative of an issue with a catheter of the patient.

In accordance with an eighth aspect of the present disclosure, which may be used in combination with any other aspect listed herein unless stated otherwise, the record of the prescribed therapy or program for the patient includes at least one of a fill threshold or a drain threshold, and the treatment data includes at least one of a fluid fill time or a fluid drain time for the treatment. The analytics processor is configured to generate an alert if the at least one of the fluid fill time or the fluid drain time exceeds the respective at least one of the fill threshold or the drain threshold.

In accordance with a ninth aspect of the present disclosure, which may be used in combination with any other aspect listed herein unless stated otherwise, the fluid fill time includes an average fluid fill time for cycles of the treatment and the fluid drain time includes an average fluid drain time for cycles of the treatment.

In accordance with a tenth aspect of the present disclosure, which may be used in combination with any other aspect listed herein unless stated otherwise, a system for predicting patient stoppage of a prescribed treatment that is administered by an automated peritoneal dialysis (“APD”) machine includes a memory device storing a training data set including treatment data and patient data for a group of patients. The training data also includes an indication as to whether the patients stopped treatments of a prescribed therapy or program. The memory device also stores at least one patient predictive model that is formed using the training data set. The at least one patient predictive model includes inputs of at least (i) counts or frequency of alerts generated by an APD machine, (ii) information related to peritoneal dialysis cycles, (iii) patient blood pressure values, and (iv) patient weight values. The memory device further stores patient data and previous treatment data for a target patient that is undergoing a prescribed therapy or program. The system also includes an interface device communicatively coupled to the APD machine via a network. The interface is configured to receive the treatment data from the APD machine for a target patient. The system further includes a predictive processor communicatively coupled to the interface device and the memory device. The predictive processor is configured to store the treatment data received by the interface device to the memory device, determine a concern score for the target patient by applying the patient data, the treatment data, and the previous treatment data of the target patent to the at least one patient predictive model, and cause the concern score to be displayed within a user interface on a clinician device.

In accordance with an eleventh aspect of the present disclosure, which may be used in combination with any other aspect listed herein unless stated otherwise, the concern score is indicative of a probability that the target patient will at least one of end treatments or reduce a frequency of treatments of the prescribed therapy or program.

In accordance with a twelfth aspect of the present disclosure, which may be used in combination with any other aspect listed herein unless stated otherwise, the predictive processor is configured to identify most significant concern parameters that contributed to the concern score, and cause an indication of the most significant concern parameters to be displayed within the user interface on the clinician device.

In accordance with a thirteenth aspect of the present disclosure, which may be used in combination with any other aspect listed herein unless stated otherwise, the memory device includes a data structure that relates medical fluid delivery recommendations to a range of concern scores.

In accordance with a fourteenth aspect of the present disclosure, which may be used in combination with any other aspect listed herein unless stated otherwise, the system further includes a guidance processor communicatively coupled to the memory device. The guidance processor is configured to compare the concern score of the target patient to the range of concern scores, select at least one recommendation based on the comparison, and cause the at least one recommendation to be displayed within the user interface on the clinician device.

In accordance with a fifteenth aspect of the present disclosure, which may be used in combination with any other aspect listed herein unless stated otherwise, a method for managing patient adherence to a prescribed therapy or program that is administered by a dialysis machine includes receiving, in an interface device, treatment data from the dialysis machine. The treatment data is indicative of a treatment duration, a fluid dwell time, and date for dialysis treatments performed by the dialysis machine according to a prescribed treatment or program. The method also includes determining, via an analytics processor communicatively coupled to the interface device, dialysis treatment parameter values by comparing the treatment data to a record of the prescribed therapy or program. The record includes a schedule of days a treatment is to be provided, a prescribed treatment duration, and an estimated prescribed treatment dwell time. The dialysis treatment parameter values include at least two of a lost treatment time parameter value as a difference or ratio between the prescribed treatment duration and the treatment durations of the dialysis treatments, a lost dwell time parameter value as a difference or ratio between the estimated prescribed treatment dwell time and the fluid dwell time of the dialysis treatments, or a completed treatment days parameter value as a difference or ratio between the schedule of days the treatment is to be provided and the date of the dialysis treatments. The method further comprises causing, via the analytics processor, the at least two of the lost treatment time parameter value, the lost dwell time parameter value, or the completed treatment days parameter value to be displayed within a user interface on a clinician device.

In accordance with a sixteenth aspect of the present disclosure, which may be used in combination with any other aspect listed herein unless stated otherwise, the method further includes storing, via the analytics processor to a memory device, the received treatment data and the record.

In accordance with a seventeenth aspect of the present disclosure, which may be used in combination with any other aspect listed herein unless stated otherwise, the dialysis machine includes an automated peritoneal dialysis (“APD”) machine or a hemodialysis machine.

In accordance with an eighteenth aspect of the present disclosure, which may be used in combination with any other aspect listed herein unless stated otherwise, the record of the prescribed therapy or program for the patient includes at least one of a fill threshold or a drain threshold, and the treatment data includes at least one of a fluid fill time or a fluid drain time for the treatment.

In accordance with a nineteenth aspect of the present disclosure, which may be used in combination with any other aspect listed herein unless stated otherwise, the method further includes at least one of generating, via the analytics processor, an alert if the at least one of the fluid fill time, a weekly average of fluid fill times, the fluid drain time, or a weekly average of fluid drain times exceeds the respective at least one of the fill threshold or the drain threshold, or generating, via the analytics processor, an alert if the at least one of the fluid fill time or the fluid drain time exceeds the respective at least one of the fill threshold or the drain threshold.

In accordance with a twentieth aspect of the present disclosure, which may be used in combination with any other aspect listed herein unless stated otherwise, the alert is indicative of an issue with a catheter of the patient.

In accordance with a twenty-first aspect of the present disclosure, which may be used in combination with any other aspect listed herein unless stated otherwise, the method further includes accessing, via the analytics processor, a data structure that relates medical fluid delivery recommendations to at least one of a range of lost treatment time parameter values, a range of lost dwell time parameter values, or a range of completed treatment days parameter values, comparing, via the analytics processor, at least one of the lost treatment time parameter value, the lost dwell time parameter value, or the completed treatment days parameter value for the patient to the respective at least one of the range of lost treatment time parameter values, the range of lost dwell time parameter values, or the range of completed treatment days parameter values, selecting, via the analytics processor, at least one recommendation based on the comparison, and causing, via the analytics processor, the at least one recommendation to be displayed within the user interface on the clinician device.

In a twenty-second aspect of the present disclosure, any of the structure, functionality, and alternatives disclosed in connection with any one or more of FIGS. 1 to 20 may be combined with any other structure, functionality, and alternatives disclosed in connection with any other one or more of FIGS. 1 to 20.

In light of the present disclosure and the above aspects, it is therefore an advantage of the present disclosure to provide an improved medical fluid delivery system.

It is another advantage of the present disclosure to determine patient adherence to a prescribed therapy or program to prevent patients from prematurely ending treatments administered by a medical fluid delivery machine.

It is a further advantage of the present disclosure to predict patient adherence to a prescribed therapy or program using a training data set of patients undergoing similar prescribed therapies or programs.

It is a further advantage of the present disclosure to provide improved clinician or caregiver guidance and efficiency.

It is still a further advantage of the present disclosure to provide improved patient outcomes from dialysis treatments.

It is yet another advantage of the present disclosure to provide a medical fluid data transfer system and methodology that may be applied to different types of medical fluid delivery machines.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic view illustrating a medical fluid data transfer system that includes at least one medical fluid delivery machine, according to an embodiment of the present disclosure.

FIG. 2 is a schematic illustration of a medical fluid delivery machine, according to an embodiment of the present disclosure.

FIG. 3 is a schematic illustration of the medical fluid data transfer system of FIG. 1 including a clinician server, according to an embodiment of the present disclosure.

FIG. 4 is a diagram of the clinician server of FIG. 3 configured for analytic processing of treatment data from a medical fluid delivery machine, according to an example embodiment of the present disclosure.

FIG. 5 is a diagram that is illustrative of at least some computations performed by the clinician server of FIGS. 3 and 4, according to an example embodiment of the present disclosure.

FIG. 6 is a diagram of an example dashboard user interface provided by the clinician server of FIGS. 3 and 4, according to an example embodiment of the present disclosure.

FIGS. 7 to 9 are diagrams of example patient adherence analytics user interfaces provided by the clinician server of FIGS. 3 and 4, according to example embodiments of the present disclosure.

FIGS. 10 and 11 are diagrams of example catheter analytics user interfaces provided by the clinician server of FIGS. 3 and 4, according to example embodiments of the present disclosure.

FIGS. 12 and 13 are diagrams of example alarm analytics user interfaces provided by the clinician server of FIGS. 3 and 4, according to example embodiments of the present disclosure.

FIG. 14 is a diagram of an example patient adherence user interface with recommendations provided by the clinician server of FIGS. 3 and 4, according to an example embodiment of the present disclosure.

FIG. 15 is a flow diagram of an example procedure to determine patient adherence, catheter, and alarm analytic data based on treatment data from a medical fluid delivery machine, according to an example embodiment of the present disclosure.

FIG. 16 is a diagram of the clinician server of FIG. 3 configured for predictive processing of treatment data from a medical fluid delivery machine, according to an example embodiment of the present disclosure.

FIG. 17 is a diagram of a concern score user interface provided by the clinician server of FIG. 16, according to an example embodiment of the present disclosure.

FIG. 18 is another diagram of a concern score user interface, according to an example embodiment of the present disclosure.

FIG. 19 is a diagram of an example patient concern score user interface with recommendations provided by the clinician server of FIG. 16, according to an example embodiment of the present disclosure.

FIG. 20 is a flow diagram of an example procedure to predict patient adherence using treatment data from a medical fluid delivery machine, according to an example embodiment of the present disclosure.

### DETAILED DESCRIPTION

A medical fluid delivery system is disclosed herein. The example medical fluid delivery system is configured to improve a patient's adherence to one or more prescribed therapies and/or programs using treatment data from a medical fluid delivery machine. The example medical fluid delivery system determines, for example, adherence analytics, which are provided to a clinician device of a clinician that is treating a patient. The adherence information includes information that is indicative as to how well a patient is adhering a prescribed therapy over time. The adherence information may include a number of days in which a prescribed therapy was completed by the patient. The adherence information may also include lost treatment time and/or low fluid dwell time that may result from a patient prematurely ending a prescribed therapy during a treatment. A clinician may use the adherence information to determine if changes to the prescribed therapy are needed and/or if patient intervention/education is needed.

In some embodiments, the adherence information may also include catheter analytic information, which compares drain and fill times from administered treatments to specified and/or threshold drain and fill times for a prescribed therapy. A deviation of the drain and fill times from the specified and/or threshold times may be indicative of a patient's catheter being improperly positioned or aligned. The deviation may also be indicative of patient constipation or a partial blockage of the catheter from fibrin strands. A clinician may use the catheter analytic information to determine if a patient's treatment adherence is affected by catheter performance, and take corrective actions with respect to the catheter (such as replacement or repositioning).

In some embodiments, the adherence information may further include alarm analytic information, which provides a summary of alarm types and alarm generation frequency. A clinician may use the alarm information to identify issues a patient may be experiencing. The alarm analytic information may also be used to reinforce the catheter analytic information and/or the treatment adherence information. For instance, the alarms may be indicative that a patient is bypassing fill and/or drain times mid-treatment to prematurely end a treatment.

The medical fluid delivery system in some embodiments may use one or more evidence based models to determine recommendations and/or guidance for improving patient adherence. The recommendations and/or guidance may be based on official guidance provided by, for example, the International Society for Peritoneal Dialysis (“ISPD”), the National Kidney Foundation (“NKF”), and/or the NKF Disease Outcomes Quality Initiative (“DOQI”). The medical fluid delivery system compares adherence analytics for each patient to a data structure that relates adherence analytic values to certain recommendations and/or guidelines. The medical fluid delivery system uses the comparison to automatically transmit recommendations to a clinician and/or a patient.

The medical fluid delivery system in some embodiments may use one or more patient predictive models configured to identify patients that are deemed a high risk to reduce or stop treatments within a week to a month ahead of time. The patient predictive models may include AI models and/or machine learning models that are trained using previous treatment data for substantially all patients that are related to a medical fluid delivery system. In addition to identifying at risk patients, the patient predictive models determine at least three to five top contributing factors or attributes that influenced the analysis. A clinician may use knowledge of the contributing factors to determine interventional actions or risk mitigation steps to improve the patient's adherence to the prescribed therapy or program. In some instances, the medical fluid delivery system uses one or more evidence based models to automatically determine recommendations or guidelines for a clinician and/or a patient based on the identified contributing factors and/or risk score.

Reference is made herein to prescribed therapies or programs and corresponding treatments. A prescribed therapy or program corresponds to one or more parameters that define how a medical fluid delivery machine is to operate to administer a treatment to a patient. For a peritoneal dialysis therapy, the parameters may specify an amount (or rate) of fresh dialysis fluid to be pumped into a peritoneal cavity of a patient, an amount of time the fluid is to remain in the patient's peritoneal cavity (i.e., a dwell time), and an amount (or rate) of used dialysis fluid and ultrafiltration (“UF”) that is to be pumped or drained from the patient after the dwell period expires. For a treatment with multiple cycles, the parameters may specify the fill, dwell, and drain amounts for each cycle and the total number of cycles to be performed during the course of a treatment (where one treatment is provided per day or separate treatments are provided during the daytime and during nighttime). In addition, the parameters (or device programs) may specify dates/times/days (e.g., a schedule) in which treatments are to be administered by the medical fluid delivery machine. Further, parameters of a prescribed therapy may specify a total volume of dialysis fluid to be administered for each treatment in addition to a concentration level of the dialysis fluid, such as a dextrose level.

While a prescribed therapy may specify parameters for each treatment provided by a medical fluid delivery machine, the treatment data reported by the machine may differ. As discussed herein, treatment data refers to data generated by a medical fluid delivery machine that is indicative of measured, detected, or determined parameter values. For example, while a prescribed therapy may specify that a treatment is to comprise five separate cycles, each with a 45 minute dwell time, a medical fluid delivery machine may administer a treatment where fewer cycles are provided, each with a 30 minute dwell time. The difference from the prescribed parameters may be due to a patient overriding the therapy program or stopping a treatment prematurely. The medical fluid delivery machine monitors how the treatment is administered and accordingly provides parameters that are indicative of the operation. The parameters for the treatment data may include, for example, a total amount of dialysis fluid administered to a patient, a number of cycles operated, a fill amount per cycle, a dwell time per cycle, a drain time/amount per cycle, an estimated amount of UF removed, a treatment start time/date, and/or a treatment end time/date. The treatment data may also include calculated parameters, such as a fill rate and a drain rate, determined by dividing the amount of fluid pumped by the time spent pumping. The treatment data may further include an identification of an alarm that occurred during a treatment, a duration of the alarm, a time of the alarm, an event associated with the alarm, and/or an indication as to whether the issue that caused the alarm was resolved or whether the alarm was silenced.

In addition to treatment data, the medical fluid delivery system may use patient data. As disclosed herein, patient data may be determined from treatment data. For example, the medical fluid delivery system may determine a patient's experience level with a medical fluid delivery machine based on the dates of treatment data. Additionally or alternatively, the patient data may include demographic data that may be provided by a clinician/patient, specified within a prescribed therapy or program, and/or provided via patient registration. The demographic data may include a patient age, a gender, a patient mobility level, a patient's renal condition, a prescription history, etc. In some embodiments, the patient data and/or the treatment data may include an identifier, which enables the medical fluid delivery system to store the received data in an appropriate patient record located in a database. The identifier may include a patient identifier, a patient name, and/or an identifier of the medical fluid delivery system.

Reference is also made herein to analytic data. As discussed in more detail below, analytic data refers to treatment data that has been compared to one or more parameters of a prescribed therapy or program and/or compared to specified thresholds. The analytic data may represent a difference between a parameter value of the treatment data and a corresponding parameter value specified in a prescribed therapy. The analytic data may also represent a ratio between certain parameters of the treatment data and corresponding parameters from a prescribed therapy and/or program (or specified limits/thresholds). The analytic data may represent a comparison for a single treatment or trends over time for a plurality of treatments. The analytic data may also represent an output from one or more patient predictive models that use AI or machine learning to determine factors that may be indicative that a patient may stop treatments or significantly reduce their administration of treatments for a prescribed therapy or program.

As discussed herein, the medical fluid delivery machine is located in a residence of a patient. However, in some embodiments, the medical fluid delivery machine may be located at a full-service medical facility and/or a self-service medical facility. In some embodiments, a patient may use a first medical fluid delivery machine that is located at their residence and use a second medical fluid delivery machine that is located at a self-service medical facility. In these embodiments, the medical fluid delivery system is configured to combine the treatment data from the first and second medical fluid delivery machines for at least one of the adherence analytics, catheter analytics, alarm analytics, and/or patient predictive analytics. In some instances, the medical fluid delivery system is configured to provide a breakdown of the reported data by medical fluid delivery machine and/or by home-based treatments compared to facility-based treatments.

### I. MEDICAL FLUID DELIVERY SYSTEM EMBODIMENT

The example medical fluid delivery system disclosed herein includes one or more medical fluid delivery machines. One example of a medical fluid delivery machine is a renal failure therapy machine. Regarding renal failure therapy machines, due to various causes, a patient's renal system can fail. Renal failure produces several physiological derangements. For instance, a patient experiencing renal failure can no longer balance water and minerals or excrete daily metabolic load. Toxic end products of nitrogen metabolism (urea, creatinine, uric acid, and others) can accumulate in the patient's blood and tissue.

Kidney failure and reduced kidney function have been treated with dialysis. Dialysis removes waste, toxins and excess water from the body that normal functioning kidneys would otherwise remove. Dialysis treatment for replacement of kidney functions is critical to many people because the treatment is life saving.

One type of kidney failure therapy is Hemodialysis (“HD”), which in general uses diffusion to remove waste products from a patient's blood. A diffusive gradient occurs across a semi-permeable dialyzer between a patient's blood and an electrolyte solution, called dialysate or dialysis fluid, to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment (typically ten to ninety liters of such fluid). The substitution fluid and the fluid accumulated by the patient in between treatments is ultrafiltered over the course of the HF treatment, providing a convective transport mechanism that is particularly beneficial in removing middle and large molecules (in hemodialysis there is a small amount of waste removed along with the fluid gained between dialysis sessions, however, the solute drag from the removal of that ultrafiltrate is not enough to provide convective clearance).

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD (HF, HDF) treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that frequent treatments remove more toxins and waste products than a patient receiving less frequent, but perhaps longer treatments. A patient receiving treatments more frequently does not experience as much of a down cycle compared to an in-center patient, who has built-up two or three days” worth of toxins prior to treatment. In certain areas, the closest dialysis center can be many miles from the patient's home causing door-to-door treatment time to consume a large portion of the day. HHD in contrast may take place overnight or during the day while the patient relaxes, works or is otherwise productive.

Another type of kidney failure therapy is peritoneal dialysis, which infuses a dialysis solution, also called dialysis fluid, into a patient's peritoneal cavity via a catheter. The dialysis fluid contacts the peritoneal membrane of the peritoneal cavity. Waste, toxins and excess water pass from the patient's bloodstream, through the peritoneal membrane and into the dialysis fluid due to diffusion and osmosis, i.e., an osmotic gradient occurs across the membrane. An osmotic agent in dialysis provides the osmotic gradient. The used or spent dialysis fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), and tidal flow dialysis and continuous flow peritoneal dialysis (“CFPD”). CAPD is a manual dialysis treatment. Here, the patient manually connects an implanted catheter to a drain to enable used or spent dialysate fluid to drain from the patient's peritoneal cavity. The patient then connects the catheter to a bag of fresh dialysis fluid to infuse fresh dialysis fluid through the catheter and into the patient. The patient disconnects the catheter from the fresh dialysis fluid bag and enables the dialysis fluid to dwell within the peritoneal cavity, where the transfer of waste, toxins, and excess water takes place. After a dwell period, the patient repeats the manual dialysis procedure, for example, four times per day, each treatment lasting between an hour and six hours. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to perform the treatment cycles manually and from having to transport supplies during the day. APD machines connect fluidly to an implanted catheter, to a source or bag of fresh dialysis fluid and to a fluid drain. APD machines pump fresh dialysis fluid from a dialysis fluid source, through the catheter and into the patient's peritoneal cavity. APD machines also allow for the dialysis fluid to dwell within the cavity and for the transfer of waste, toxins, and excess water to take place. The source may include multiple sterile dialysis fluid bags.

APD machines pump used or spent dialysate from a patient's peritoneal cavity, though a catheter, and to a drain. As with the manual process, several drain, fill and dwell cycles occur during dialysis. A “last fill” occurs at the end of APD and remains in the peritoneal cavity of the patient until the next treatment.

Any of the above modalities performed by a machine may be run on a scheduled basis and may require a start-up procedure. For example, dialysis patients typically perform treatment on a scheduled basis specified by a prescribed therapy or program, such as every other day, daily, etc. Blood treatment machines typically require a certain amount of time before treatment for setup, for example, to run a disinfection procedure. Patients for the above modalities may lead busy lives and have projects to perform or errands to run on a day scheduled for treatment.

Much of the appeal of a home treatment for the patient revolves around the lifestyle flexibility provided by allowing a patient to perform treatment in his or her home largely according to his or her own schedule. The home medical fluid delivery machine may, however, include software timers that dictate to and constrain the patient. A home hemodialysis system may, for example, require a patient to be in immediate proximity to the home hemodialysis machine to initiate pre-treatment, during treatment, and post-treatment sequences.

In one particular example, a therapy machine may reuse certain components by disinfecting them in between treatments. The machine may employ one or more disinfection timer that requires a patient or caregiver to start a treatment using the machine before the disinfection timer expires. Otherwise, the patient will have to wait until another disinfection procedure is completed before starting treatment. The therapy machine in an embodiment communicates the treatment start time deadlines via the machine's graphical user interface.

It is contemplated for the software of the system and methodology of the present disclosure to disable communication between the patient and/or caregiver and the machine whenever the machine is in a “patient connected” software state. For example, if a clinician tries to send a command to a machine currently treating a patient, the command may be intercepted by a middleware software application so that the command is not transferred to the machine. The middleware software application may then communicate back to the clinician informing that the machine is busy and not accepting communication.

The examples described herein are applicable to any medical fluid delivery system that delivers a medical fluid, such as blood, dialysis fluid, substitution fluid or and intravenous drug (“IV”). The examples are particularly well suited for kidney failure therapies, such as all forms of hemodialysis (“HD”), hemofiltration (“HF”), hemodiafiltration (“HDF”), continuous renal replacement therapies (“CRRT”) and peritoneal dialysis (“PD”), referred to herein collectively or generally individually as a prescribed therapy or program. The medical fluid delivery machines may alternatively be a drug delivery or nutritional fluid delivery device, such as a large volume peristaltic type pump or a syringe pump. The machines described herein may be used in home settings. For example, a machine operating with a data transfer component may be employed with a home HD machine, which can, for example, be run at night while the patient is sleeping. The medical fluid data transfer system and methodology of the present disclosure may alternatively be used to help clinicians or nurses in hospitals and/or clinics.

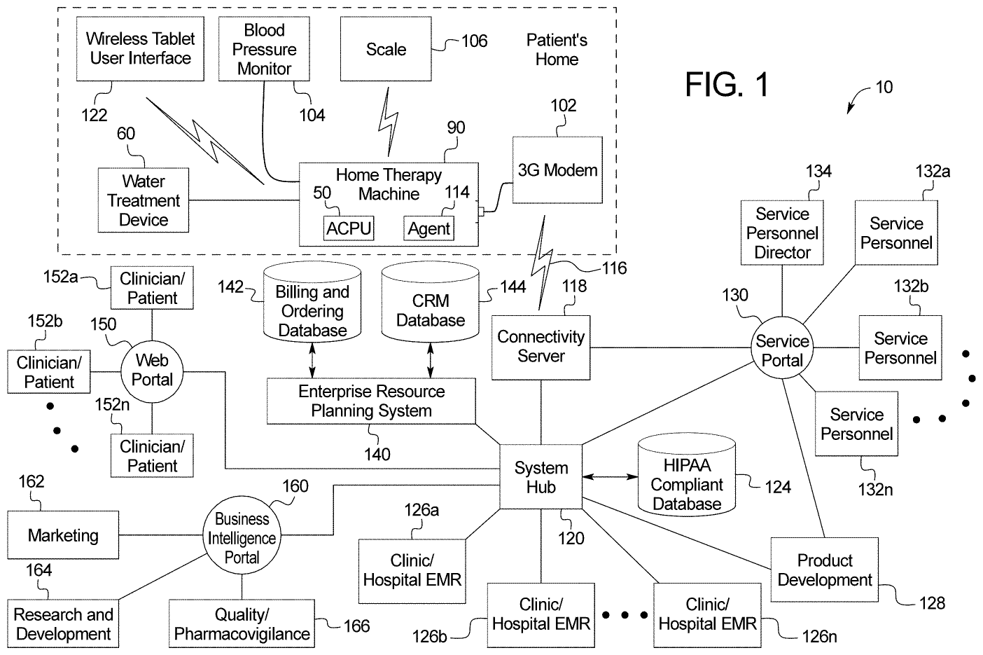

Referring now to the drawings, and in particular to FIG. 1, a medical fluid data transfer system 10 is illustrated. The example system 10 includes many medical fluid delivery machines 90 (one type of which is discussed in detail below). The machines 90 of the medical fluid data transfer system 10 may be of a same type (e.g., all PD machines) or be of different types (e.g., a mix of HD, PD, CRRT, and medical or nutritional fluid delivery).

While a single medical fluid delivery 90 is illustrated as communicating with a connectivity server 118, the system 10 manages the operation of a plurality of medical fluid delivery systems and machines, of the same type or of different types listed above. For example, there may be M number of hemodialysis machines 90, N number of hemofiltration machines 90, O number of CRRT machines 90, P number of peritoneal dialysis machines 90, Q number of home drug delivery machines 90, and R number of nutritional or drug delivery machines 90 connected to the server 118 and operating with the system 10. The numbers M through R may be the same or different numbers, and may be zero, one, or more than one. In FIG. 1, the medical fluid delivery machine 90 is illustrated as a therapy machine 90 (the home indicated by dashed lines).

The therapy machine 90 may receive at its front end purified water from a water treatment device 60. The water treatment device 60 connects to the therapy machine 90 via an Ethernet cable, in an embodiment. The therapy machines 90 in the illustrated embodiment operates with other devices besides the water treatment device 60, such as a blood pressure monitor 104, a weigh scale, e.g., a wireless weigh scale 106, and a user interface such as a wireless tablet user interface 122. The therapy machine 90 connects to the server 118 wirelessly in one embodiment via a modem 102. Each of these components may (but does not have to be) located within the patient's home, as demarcated by the dashed lines in FIG. 1. Any one, or more, or all of the components 60, 104, 106 and 122 may communicate wired or wirelessly with the therapy machine 90. Wireless communication may be via Bluetooth™, WiFi™, Zigbee®, Z-Wave®, wireless Universal Serial Bus (“USB”), infrared, or any other suitable wireless communication technology. Alternatively, any one, or more or all of the components 60, 104, 106 and 122 may communicate with the therapy machine 90 via wired communication.

The example connectivity server 118 communicates with the medical fluid delivery machine 90 via a medical device system hub 120. The example system hub 120 enables data and information concerning each therapy machine 90 and its peripherals to travel back and forth via the connectivity server 118 between the machines 90 and the other devices that are connected to the server 118. In the illustrated embodiment, the system hub 120 is connected to a service portal 130, an enterprise resource planning system 140, a web portal 150, a business intelligence portal 160, a HIPAA compliant database 124, a product development team 128, and electronic medical records databases maintained, for example, at clinics or hospitals 126a to 126n. The connectivity server 118 and/or the portals 130, 150, and 160 may include gateway devices.

The illustrated electronic medical records (“EMR”) databases may be located at clinics or hospitals 126a to 126n and store electronic information concerning patients. The system hub 120 may transmit the data collected from log files of machine 90 (e.g., treatment data) to the hospital or clinic databases 126a to 126n to merge or supplement that patient's medical records. The databases at clinics or hospitals 126a to 126n may contain patient-specific treatment and prescription data (e.g., prescribed therapies or programs), where access to such databases may be highly restricted. The example enterprise resource planning system 140 is configured to obtain and compile data generated via patient and clinician website access, such as complaints, billing information, and life cycle management information. The web portal 150 enables patients and clinics 152a to 152n treating the patients to access a website that is publicly available. The business intelligence portal 160 collects data from the system hub 120 and provides the data to marketing 162, research and development 164, and quality/pharmacovigilance 166.

It should be appreciated that the systems, methods and procedures described herein may be implemented using one or more computer programs or components. The programs of the components may be provided as a series of computer instructions on any computer-readable medium, including random access memory (“RAM”), read only memory (“ROM”), flash memory, magnetic or optical disks, optical memory, or other storage media. The instructions may be configured to be executed by a processor, which when executing the series of computer instructions, performs or facilitates the performance of all or part of the disclosed methods and procedures that are described herein.

In one embodiment, the therapy machine 90 performs a home treatment, such as home peritoneal dialysis on a patient at the patient's home, and then reports the results of that treatment (as treatment data) to the system hub 120, which may be in communication with one or more servers. As described in more detail below, the one or more servers analyze the treatment data for reporting to clinicians, doctors, and/or nurses who are responsible for managing the health and well-being of that patient.

The therapy machines 90 in an embodiment writes log files using, e.g., a Linux™ operating system. The log files document pertinent therapy machine 90 data, including peripheral device data. The log files may include any one or more of Extensible Markup Language (“XML”), comma-separated values (“CSV”), or text files. The log files are placed into a file server repository managed by software of therapy machine 90. It is also contemplated to store data at a peripheral device, e.g., water treatment device 60, which is not sent to machine 90. Such data may otherwise be obtained via the wired or wireless connection to the peripheral device or downloaded through other data connections or storage media. For example, a service technician can access additional data via a laptop connected to the water treatment device 60 or the wireless weigh scale 106, e.g., via an Ethernet connection. Alternatively, the additional data may be retrieved remotely from the peripheral devices, with the therapy machine 90 serving as the data transfer liaison between the peripheral device and authorized clients of medical fluid data transfer system.

In one embodiment, the therapy machine 90, e.g., via the internet, uses a connectivity service to transfer treatment data between the modem 102 and the system hub 120. Here, a dedicated line may be provided at each patient's home for connecting the therapy machine 90 to the connectivity server 118 via the modem 102. The therapy machine 90, in one embodiment, accesses the internet using a separate, e.g., 3G, 4G or 5G, modem 102. The modem 102 may use an internet Service Provider (“ISP”), such as Vodafone™. In one implementation, a connectivity agent 114 developed by a connectivity service provider (e.g., provider of connectivity server 118) is installed onto the therapy machine 90 and run on a primary control processor (“ACPU”) 50 of the machine. One suitable connectivity service is provided by Axeda™, which provides a secure managed connection 116 between medical devices and the connectivity server 118.

The example connectivity agent 114 of FIG. 1 is configured to enable the therapy machine 90 to connect to the connectivity server 118 and transfer the treatment data to and from the connectivity server 118. A connectivity service operating via the agent 114 and the server 118 ensures that the connection with the machine 90 is secure, ensures that the data correctly passes through the machine 90's firewalls, detects whether there has been a data or system crash, and ensures that the connectivity server 118 is communicating with the correct therapy machine 90.

In one embodiment, the therapy machine 90 may only connect to the connectivity server 118 when the connectivity agent 114 is turned on or activated. During treatment and post-treatment disinfection, while the machine 90 and its peripherals are functioning, the connectivity agent 114 is automatically turned off in one embodiment, which prevents the therapy machine 90 from communicating with any entity and sending or receiving data during treatment and disinfection or when machine 90 is live or running. When the therapy machine 90 is idle, e.g., after treatment and post-disinfection is complete, the ACPU 50 turns the connectivity agent 114 on, in one embodiment. In an embodiment, the connectivity agent 114 is off during treatment, and possibly pre-treatment. After treatment, the connectivity agent 114 retrieves the log files from the therapy machine 90 and transfers the treatment data to the connectivity server 118 using the connectivity service. The connectivity service routes data packets to their proper destination, but in one embodiment, does not modify, access, or encrypt the data.

In medical fluid data transfer system 10 system of FIG. 1, the connectivity service via the connectivity server 118 may communicate data to various places via the system hub 120, such as the service portal 130, the clinics or hospitals 126a to 126n, and the web portal 150. The connectivity server 118 enables service personnel 132a to 132n and/or clinicians to track and retrieve various assets across the network, such as appropriate therapy machines 90 and 3G, 4G or 5G modem 102, and their associated information, including machine or modem serial numbers. The connectivity server 118 may also be used to receive and provide firmware upgrades, approved by a director of service personnel 134 and obtained remotely via the service portal 130, to the authorized therapy machines 90 and associated peripherals, such as water treatment devices 60.

### A. Example Medical Fluid Delivery Machine

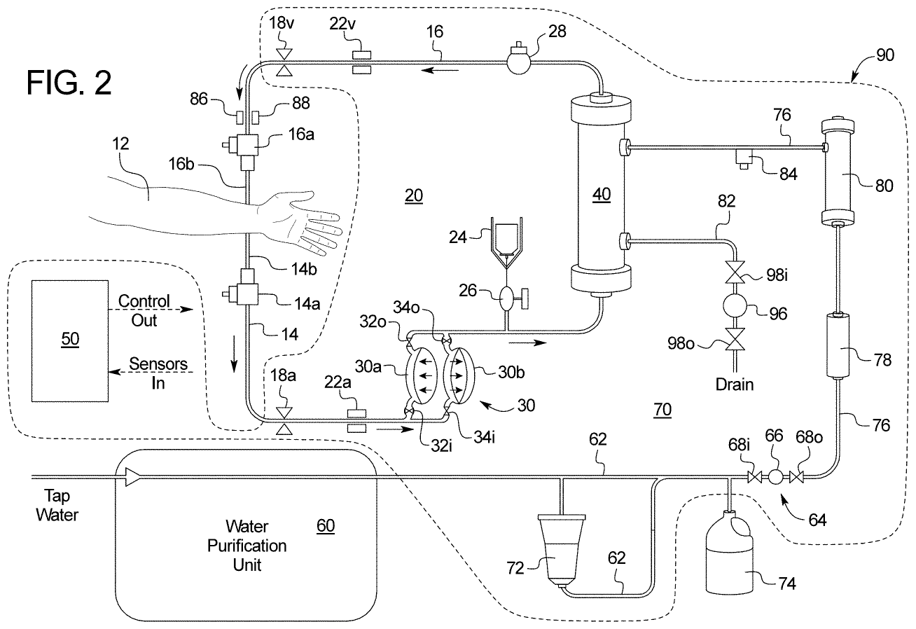

Referring now to FIG. 2, an example of an HD flow schematic for the medical fluid delivery machine 90 is illustrated. Because the HD system of FIG. 2 is relatively complicated, FIG. 2 and its discussion also provide support for any of the renal failure therapy modalities discussed above and for an IV, drug delivery, or nutritional fluid delivery machine. Generally, the medical fluid delivery machine 90 is shown having a simplified version of a dialysis fluid or process fluid delivery circuit. The blood circuit is also simplified, but not to the degree that the dialysis fluid circuit is simplified. It should be appreciated that the circuits have been simplified to make the description of the present disclosure easier, and that the systems, if implemented, would have additional structure and functionality, such as is found in the publications incorporated by reference above.

The medical fluid delivery machine 90 of FIG. 2 includes a blood circuit 20. The example blood circuit 20 pulls blood from and returns blood to a patient 12. Blood is pulled from a patient 12 via an arterial line 14, and is returned to the patient via a venous line 16. The arterial line 14 includes an arterial line connector 14a that connects to an arterial needle 14b, which is in blood draw communication with the patient 12. The venous line 16 includes a venous line connector 16a that connects to a venous needle 16b, which is in blood return communication with the patient. The arterial and venous lines 14 and 16 also include line clamps 18a and 18v, which can be spring-loaded, fail-safe mechanical pinch clamps. The line clamps 18a and 18v are closed automatically in an emergency situation in one embodiment.

The arterial and venous lines 14 and 16 also include air or bubble detectors 22a and 22v, respectively, which can include ultrasonic air detectors. The air or bubble detectors 22a and 22v are configured to detect air in the arterial and venous lines 14 and 16, respectively. If air is detected by one of the air detectors 22a and 22v, the medical fluid delivery machine 90 closes line clamps 18a and 18v, pauses the blood and dialysis fluid pumps, and provides instructions to the patient to clear the air so that treatment can resume.

A blood pump 30 is located in the arterial line 14 in the illustrated embodiment. The blood pump 30 includes a first blood pump pod 30a and a second blood pump pod 30b. The blood pump pod 30a operates with an inlet valve 32i and an outlet valve 32o. The blood pump pod 30b operates with an inlet valve 34i and an outlet valve 34o. In an embodiment, the blood pump pods 30a and 30b are each blood receptacles that include a hard outer shell, e.g., spherical, with a flexible diaphragm located within the shell, forming a diaphragm pump. One side of each diaphragm receives blood, while the other side of each diaphragm is operated by negative and positive air pressure. The blood pump 30 is alternatively a peristaltic pump operating with the arterial line 14 or multiple peristaltic pumps operating with the arterial line 14 and the venous line 16.

As shown in FIG. 2, a heparin vial 24 and a heparin pump 26 are located between the blood pump 30 and a blood filter 40 (e.g., dialyzer). The heparin pump 26 may be a pneumatic pump or a syringe pump (e.g., stepper motor driven syringe pump). Supplying heparin upstream of the blood filter 40 helps to prevent clotting of the filter's membranes.

A primary control processor (“ACPU”) or control unit control unit 50 includes one or more processors and memory. The control unit 50 receives air detection signals from air detectors 22a and 22v (and other sensors of the medical fluid delivery machine 90, such as temperature sensors, blood leak detectors, conductivity sensors, pressure sensors, and access disconnection transducers 86, 88), and controls components such as line clamps 18a and 18v, blood pump 30, heparin pump 26, dialysis fluid pumps 64 and 96, and valves 32i, 32o, 34i, 34o, 68i, 68o, 98i and 98o. Blood exiting the blood filter 40 via the venous line 16 flows through an airtrap 28. The example airtrap 28 removes air from the blood before the dialyzed blood is returned to the patient 12 via venous line 16.

With the hemodialysis version of medical fluid delivery machine 90 of FIG. 2, dialysis fluid is pumped along the outside of the membranes of blood filter 40, while blood is pumped through the insides of the blood filter membranes. Dialysis fluid is prepared beginning with the purification of water via a water purification unit 60. One suitable water purification unit is set forth in U.S. Patent Publication No. 2011/0197971, entitled, “Water Purification System and Method”, filed Apr. 25, 2011, the entire contents of which are incorporated herein by reference and relied upon. In an embodiment, the water purification unit 60 includes filters and other structures to purify tap water (e.g., remove pathogens and ions such as chlorine), so that the water is, in one implementation, below 0.03 endotoxin units/ml (“EU/ml”) and below 0.1 colony forming units/ml (“CFU/ml”). The water purification unit 60 may be provided in a housing separate from the housing or chassis of the hemodialysis machine 90, which includes the blood circuit 20 and the dialysis fluid circuit 70.

Dialysis fluid circuit 70 is again highly simplified in FIG. 2 to ease illustration. The dialysis fluid circuit 70 in actuality may include all of the relevant structure and functionality set forth in the publications incorporated by reference above. Certain features of dialysis fluid circuit 70 are illustrated in FIG. 2. In the illustrated embodiment, the dialysis fluid circuit 70 includes a to-blood filter dialysis fluid pump 64. The example pump 64, in one embodiment, is configured the same as blood pump 30. The pump 64, like pump 30, includes a pair of pump pods 66 each having inlet valves 68i and outlet valves 68o, which again may be spherically configured. The two pump pods, like with the blood pump 30, are operated alternatingly so that one pump pod is filled with HD dialysis fluid, while the other pump pod expels HD dialysis fluid.

The pump 64 is a to-blood filter dialysis fluid pump. There is another dual pod pump chamber 96 operating with valves 98i and 98o located in a drain line 82 to push used dialysis fluid to drain. There is a third pod pump (not illustrated) for pumping purified water through a bicarbonate cartridge 72. There may be a fourth pod pump (not illustrated) used to pump acid from an acid container 74 into a mixing line 62. The third and fourth pumps may be single pod pumps because continuous pumping is not as important in the mixing line 62 due to a buffering dialysis fluid tank (not illustrated) between the mixing line 62 and the to-blood filter dialysis fluid pump 64 in one embodiment.

A fifth pod pump (not illustrated) may be provided in drain line 82 to remove a known amount of ultrafiltration (“UF”) when an HD therapy is provided. The medical fluid delivery machine 90 keeps track of the UF pump to control and know how much ultrafiltrate has been removed from the patient. The medical fluid delivery machine 90 ensures that the necessary amount of ultrafiltrate is removed from the patient by the end of a treatment.

Each of the above-described pumps may alternatively be a peristaltic pump operating with a pumping tube. If so, the system valves may still be actuated pneumatically according to the features of the present disclosure.

In one embodiment, purified water from the water purification unit 60 is pumped along the mixing line 62 though the bicarbonate cartridge 72. Acid from the container 74 is pumped along the mixing line 62 into the bicarbonated water flowing from the bicarbonate cartridge 72 to form an electrolytically and physiologically compatible dialysis fluid solution. The pumps and temperature-compensated conductivity sensors used to properly mix the purified water with the bicarbonate and acid are not illustrated but are disclosed in detail in the publications incorporated by reference above.

FIG. 2 also illustrates that dialysis fluid is pumped along a fresh dialysis fluid line 76, through a heater 78 and an ultrafilter 80, before reaching the blood filter 40, after which used dialysis fluid is pumped to drain via the drain line 82. The heater 78 heats the dialysis fluid to body temperature or about 37° C. The ultrafilter 80 further cleans and purifies the dialysis fluid before reaching the blood filter 40, filtering foreign matter and/or contaminants introduced for example via bicarbonate cartridge 72 or acid container 74 from the dialysis fluid.

The example dialysis fluid circuit 70 also includes a sample port 84. The dialysis fluid circuit 70 may further include a blood leak detector (not illustrated but used to detect if a blood filter 40 fiber is torn) and other components that are not illustrated, such as balance chambers, plural dialysis fluid valves, and a dialysis fluid holding tank, all illustrated and described in detail in the publications incorporated by reference above.

In the illustrated embodiment, the medical fluid delivery machine 90 is an online, pass-through system that pumps dialysis fluid through the blood filter one time and then pumps the used dialysis fluid to drain. Both the blood circuit 20 and the dialysis fluid circuit 70 may be hot water disinfected after each treatment, such that the blood circuit 20 and the dialysis fluid circuit 70 may be reused. In one implementation, the blood circuit 20, including the blood filter 40, may be hot water disinfected and reused daily for about one month, while the dialysis fluid circuit 70 may be hot water disinfected and reused for about six months.

In alternative embodiments, for CRRT for example, multiple bags of sterilized dialysis fluid or infusate are grouped together and used one after another. In such a case, the emptied supply bags can serve as drain or spent fluid bags.

The medical fluid delivery machine 90 includes an enclosure as indicated by the dashed line of FIG. 2. The enclosure of the machine 90 varies depending upon the type of treatment, whether the treatment is in-center or a home treatment, and whether the dialysis fluid/infusate supply is a batch-type (e.g., bagged) or online.

In some embodiments, the medical fluid delivery machine 90 of FIG. 2 is configured to perform one or more prescribed PD therapies or programs. In these embodiments, the fresh dialysis fluid includes peritoneal dialysis dialysate comprising a solution or mixture that has between 0.5% and 10% dextrose (or more generally glucose), preferably between 1.5% and 4.25%. The peritoneal dialysis dialysate may include, for example, Dianeal®, Physioneal®, Nutrineal®, and/or Extraneal® dialysates marketed by the assignee of the present disclosure. The dialysate may additionally or alternatively include a percentage of icodextrin.

In PD embodiments, the blood circuit 20 including the blood filter is removed. Instead, the fresh dialysis fluid line 76 is connected to a catheter placed in proximity to a peritoneal cavity of the patient 12. The catheter is also connected to the drain line 82, which removes used dialysis fluid including accumulated UF. In some embodiments, the drain line 82 may be connected to a recirculation device, such as a sorbent cartridge, which is configured to cleanse the used dialysis fluid before it is returned to the patient's peritoneal cavity. In some instances, the recirculation device may remove uremic toxins from waste dialysate and re-inject therapeutic agents (such as ions and/or glucose). One commonly used sorbent is made from zirconium phosphate, which is used to remove ammonia generated from the hydrolysis of urea.

### B. Connectivity Embodiment of the Example Medical Fluid Delivery System

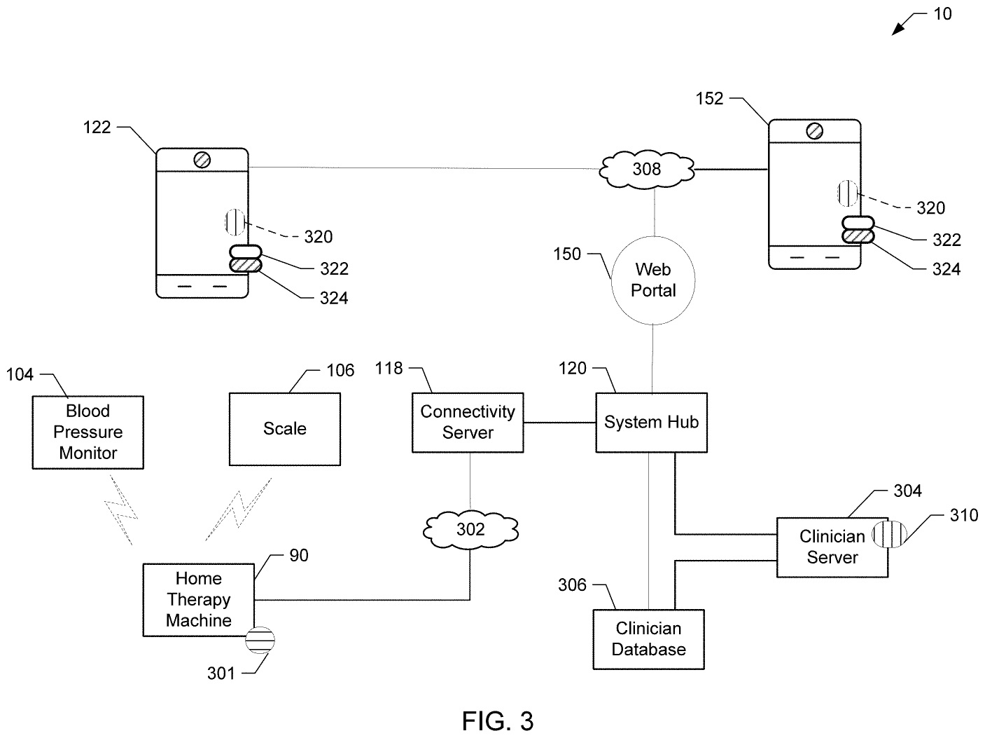

FIG. 3 illustrates a diagram of the medical fluid data transfer system 10 of FIG. 1, according to an example embodiment of the present disclosure. The example medical fluid data transfer system 10 includes, for example, a personal mobile communication device 122 that is operated by a patient, and a clinician device 152 that is operated by a clinician. The medical fluid data transfer system 10 also includes a blood pressure monitor 104, a scale 106, and a therapy machine 90 (e.g., a medical fluid delivery machine), which are similar to the respective devices discussed above in connection with FIGS. 1 and 2. The personal mobile communication device 122, the therapy machine 90, the blood pressure monitor 104, and the scale 106 may be located, for example, at a patient's home, a self-service clinic, and/or a serviced medical clinic.

The therapy machine 90 may include any type of hemodialysis machine, peritoneal dialysis machine, CRRT machine, drug and/or nutritional fluid delivery machine, and combinations thereof. The therapy machine 90 may provide, for example continuous cycling peritoneal dialysis (“CCPD”), tidal flow automated peritoneal dialysis (“APD”), and continuous flow peritoneal dialysis (“CFPD”). The therapy machine 90 may perform drain, fill, and dwell cycles automatically, typically while a patient sleeps.

The example therapy machine 90 may also include one or more control interfaces 301 for displaying instructions and receiving control inputs from a user. The control interfaces 301 may include buttons, a control panel, and/or a touchscreen. The control interfaces 301 may also be configured to enable a user to navigate to a certain window or user interface on a screen of the therapy machine 90. The control interfaces 301 may further provide instructions for operating or controlling the therapy machine 90.

The example therapy machine 90 may receive one or more prescribed therapies or programs remotely from a clinician server 304 and/or a clinician database 306. Additionally or alternatively, the therapy machine 90 may be programmed locally via the control interface 301 with a prescribed therapy or program. As discussed herein, a prescribed therapy includes parameters that specify how the therapy machine 90 is to administer one or more scheduled treatments to a patient (e.g., the patient 12 of FIG. 1). The therapy parameters may include a number of fill-dwell-drain cycles for a peritoneal dialysis therapy in addition to a duration for each phase. The therapy parameters may also include a total volume of dialysis fluid to be administered (and/or a volume of fluid to be administered per cycle), a dextrose concentration, and/or a target UF removal level. The therapy parameters may also include a schedule of treatment dates and a total treatment duration. In some embodiments, the clinician server 304 may remotely update any one of the prescribed therapy parameters.

The example blood pressure monitor 104 includes any device configured to measure a blood pressure and/or pulse of a patient. For example, the blood pressure monitor 104 may measure a patient blood pressure before, during, and/or after a renal failure therapy treatment. The blood pressure monitor 104 may display a digital value indicative of a patient's blood pressure. Alternatively, the blood pressure monitor 104 may display a physical scale with a dial that aligns with a numerical value to indicate a measured blood pressure. In some embodiments, the blood pressure monitor 104 may store blood pressure values before, during, and/or after treatment in separate windows such that patient input is required to view all the values when medical information is recorded. The blood pressure monitor 104 may be integrated with the therapy machine 90. In another embodiment, the blood pressure monitor 104 may include a wearable sensor, such as a smartwatch or fitness-tracking device. The blood pressure monitor 104 may transmit a measured blood pressure value (e.g., treatment data) via a wired or wireless connection to the therapy machine 90 and/or the personal mobile communication device 122, which is then routed the system hub 120 for processing.

The example weight scale 106 includes any device configured to measure a mass of a patient or treatment component. For example, the weight scale 106 may measure a patient weight before, during, and/or after a renal failure therapy treatment. Additionally or alternatively, the weight scale 106 may measure a supply or drain bag for tracking a renal failure therapy. Specifically, weight scale 106 may be used to measure an amount of UF removed or an amount of fluid provided to a patient. The weight scale 106 may display a digital value indicative of weight. Alternatively, the weight scale 106 may transmit a measured weight value (e.g., treatment data) via a wired or wireless connection to the therapy machine 90 and/or the personal mobile communication device 122, which is then routed the system hub 120 for processing.

Collectively, the blood pressure monitor 104 and the scale 106 are referred to as medical devices. It should be appreciated that the medical fluid data transfer system 10 may include additional medical devices such as an infusion pump (e.g., a syringe pump, a linear peristaltic pump, a large volume pump (“LVP”), an ambulatory pump, a multi-channel pump), an oxygen sensor, a respiratory monitor, a glucose meter, a blood pressure monitor, an electrocardiogram (“ECG”) monitor, and/or a heart rate monitor. In other examples, the medical fluid data transfer system 90 may include fewer medical devices and/or medical devices integrated together (e.g., a blood pressure monitor 104 integrated with the therapy machine 90).

As shown in FIG. 3, the therapy machine 90 is communicatively coupled to the connectivity server 118 via a network 302. As discussed above in connection with FIGS. 1 and 2, the connectivity server 118 provides bidirectional communication between the therapy machine 90 and the system hub 120. The network 302 may include any wired or wireless network including the Internet and/or a cellular network.

The example system hub 120 is also communicatively coupled to the clinician server 304 and the clinician database 306. As described in more detail below, the clinician server 304 is configured to execute one or more instructions, routines, algorithms, applications, or programs 310 for performing analytics on treatment data. The clinician database 306 is configured to store prescribed therapies or programs for each patient associated with the system 10. The clinician database 306 is also configured to store one or more records for each patient that include treatment data from the respective therapy machine 90 and/or patient data. The clinician database 306 may also store results of analytics performed by the clinician server 304 on the treatment and/or patient data. Further, the clinician database 306 may store a data structure that relates treatment recommendations and/or guidelines, as specified by ISPD, NKF, and/or NKF DOQI, with ranges of analytic values corresponding to patient therapy adherence, catheter operation, and/or alarms.

As illustrated in FIG. 3, the example medical fluid data transfer system 10 includes a web portal 150 to facilitate the transmission of data to the clinician device 152 and/or the personal mobile communication device 122 via a network 308. The example network 308 may include any wired and/or wireless network, such as the Internet and/or a cellular network. The web portal 150 may include one or more application programming interfaces (“API”) or other network interfaces that provide for the communication of treatment data, patient data, analytic data, and/or recommendations/guidelines. In some instances, the web portal 150 may be configured as a gateway device and/or firewall such that only authorized users and/or devices may communicate with the clinician server 304 and/or the clinician database 306. Further, the web portal 150 may create a separate session for each connected device 122 and 152.

The clinician device 152 and/or the personal mobile communication device 122 may include an application 320 that is configured to interface with the web portal 150 for communicating with the clinician server 304 and/or the clinician database 306. For example, the application 320 may include one or more use interfaces with data fields (discussed further below) that display treatment data, patient data, and/or analytic data. The data fields are mapped to one or more APIs at the web portal 150, which are linked to one or more data structures at the clinician database 306 and/or the clinician server 304. Selection of a user interface via the application 320, causes a request message to be transmitted from the application 320 to the web portal 150 identifying the data fields that are related to the requested user interface. The request message may also identify a patient. In response, the web portal 150 transmits one or more request messages to the clinician database 306 and/or the clinician server 304 to retrieve the treatment, patient, and/or analytic data associated with the identified patient and identified data fields.

In other instances, the application 320 is a web browser configured to access one or more web pages via the web portal 150 that are hosted or managed by the clinician server 304. In these other instances, the clinician server 304 provides the user interfaces and corresponding data fields in one or more web pages. A user may interact with the web browser to view or enter desired data. The application 320 may also include native control or other installed applications on the devices 122 and 152.

In some instances, the web portal 150 is configured to convert treatment data and/or analytics data from a text-based standard or Health-Level-7 (“HL7”) standard (e.g., a medical standard) to a web-based message (e.g., a HTTP message, an HyperText Markup Language (“HTML”) message, an Extensible Markup Language (“XML”) message, a JavaScript Object Notation (“JSON”) payload, etc.). In other embodiments, the connectivity server 118 is configured to convert HL7 treatment data from the therapy machine 90 into a text-based or web-based format (e.g., a JSON format) for processing by the clinician server 304 and storage by the clinician database 306.

In the illustrated example of FIG. 3, the example personal mobile communication device 122 and the clinician device 152 include a processor 322 that is in communication with a memory 324 storing instructions. At least some of the instructions define or specify the application 320, that when executed by the processor 322, cause the processor 322 to provide interfaces for displaying and interacting with treatment data, patient data, and/or analytic data stored in the clinician database 306. The processor 322 may comprise digital and/or analog circuitry structured as a microprocessor, application specific integrated circuit (“ASIC”), controller, etc. The memory 324 includes a volatile or non-volatile storage medium. Further, the memory 324 may include any solid state or disk storage medium.

### II. TREATMENT ANALYTICS EMBODIMENTS

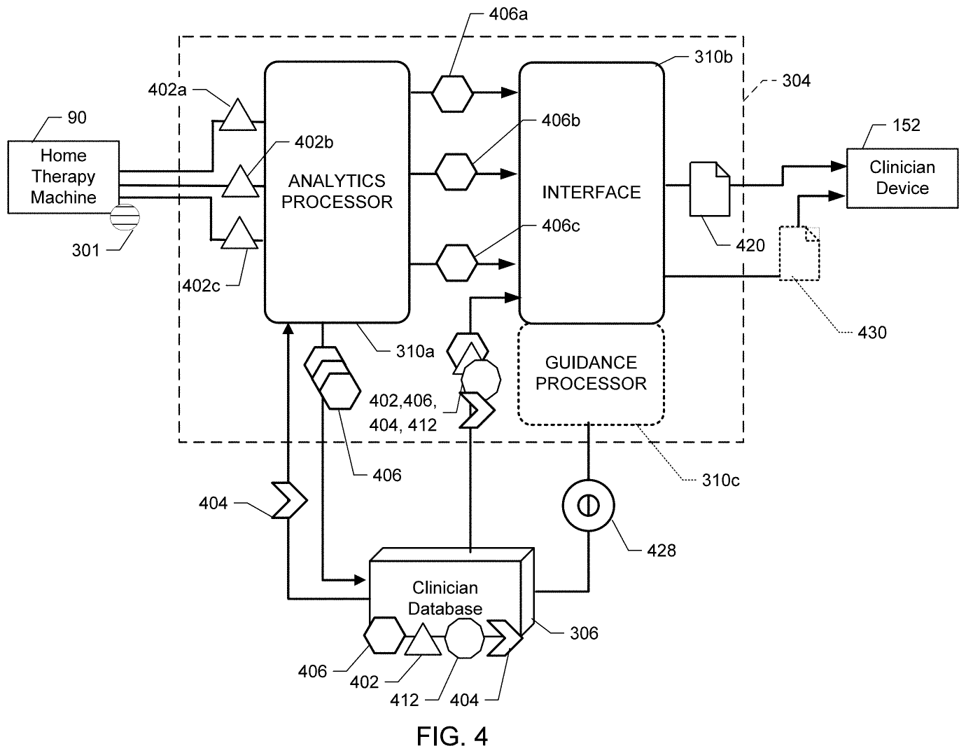

The example clinician server 304 of FIG. 3 is configured to analyze patient treatment data from therapy machines 90 (e.g., medical fluid delivery machines) to determine a patient's adherence to a prescribed therapy or program. FIG. 4 shows a diagram of the clinician server 304 according to an example embodiment of the present disclosure. In the illustrated example, the clinician server 304 includes applications 310 that are configured as an analytics processor or engine 312a and an interface 310b. In some embodiments, the applications 310 may also include a guidance processor or engine 310c. The processors 310a to 310c are defined by one or more machine-readable instructions that specify how treatment data is to be processed, stored, and accessed for display on a clinician device 152 (and/or a personal mobile communication device 122).

### A. Data Analytics Embodiment

As shown in FIG. 4, the analytics processor 310a receives treatment data 402 from the therapy machine 90. The treatment data 402 includes data 402a that is used for determining adherence, data 402b that is used for determining catheter operability, and data 402c that is used for determining alarm fatigue. The data 402 is received in one or more treatment log files from the therapy machine 90. The analytics processor 310a is configured to parse or otherwise extract the needed data from log files using data labels, metadata, or preprogrammed knowledge regarding data placement in the file. In some embodiments, the data 402 is received in the clinician database 306 from the therapy machine 90, and subsequently accessed by the analytics processor 310a.

In some embodiments, the treatment data 402 may include identifiers for storing or organizing the data in the clinician database 306. For example, the therapy machine 90 may include a device identifier with the data 402 and/or a patient identifier. The analytics processor 310a is configured to use the identifier for storing the treatment data 402 and the corresponding analytics data to the appropriate patient record in the database 306. In some instances, the analytics processor 310a may use the identifier to distinguish as to whether the treatment data 402 is from an at-therapy machine 90 or a machine 90 that is located in a clinic. The analytics processor 310a may distinguish between treatment administration locations to determine if a patient is more adherent at home or in a clinical setting.

The example clinician database 306 may store patient data 412 in addition to treatment data 402. Patient data 412 includes information that relates to a patient. The patient data 412 may include a patient identifier, a patient name, a patient age, a gender, medical conditions(s), renal state information, and/or an estimated experience level with therapy machines 90. The patient data 412 may be transmitted to the clinician database 306 from the personal mobile communication device 122 and/or the clinician device 152. In some embodiments, the patient data 412 is provided when the patient is registered with the medical fluid data transfer system 10 disclosed herein.

The treatment data 402a that is related to adherence includes an indication of a date and/or time in which a treatment was administered by the therapy machine 90. The treatment data 402a also includes a duration of the treatment, and measured dwell times for each cycle of the treatment (and/or a cumulative dwell time for all cycles). In some embodiments, the treatment data 402a may also include a total volume of dialysis solution provided to the patient, a dextrose level of the peritoneal dialysis fluid, an estimated amount of UF removed, and/or indications of therapy-related events that occurred during the treatment (such as pausing of the treatment).

The treatment data 402b that is related to catheter operability includes a fill time or rate for each cycle (and/or a cumulative fill time for all cycles). The treatment data 402b also includes a drain time or rate for each cycle (and/or a cumulative drain time for all cycles). In some instances, the analytics processor 310a is configured to calculate an average fill time and drain time for all cycles of the treatment. In some embodiments, the treatment data 402c may include an indication of any alarms that activated during a treatment due to a line occlusion during a fill or drain phase of a cycle, which may be indicative of at least a partially blocked catheter. The treatment data 402c may also include an indication of any alarms that activated during a treatment due to a fluid leak during a fill or drain phase of a cycle, which may be indicative of a misaligned catheter.

The treatment data 402c that is related to alarm fatigue includes an indication of alarms generated by the therapy machine 90 during a treatment. The treatment data 402c may also include an alarm type, a duration of the alarm, and/or an indication as to whether the condition that triggered the alarm was rectified or whether the patient silenced or bypassed the alarm. The treatment data 402c may further include an indication as to whether an alarm was escalated or whether a treatment was terminated as a result of an alarm.

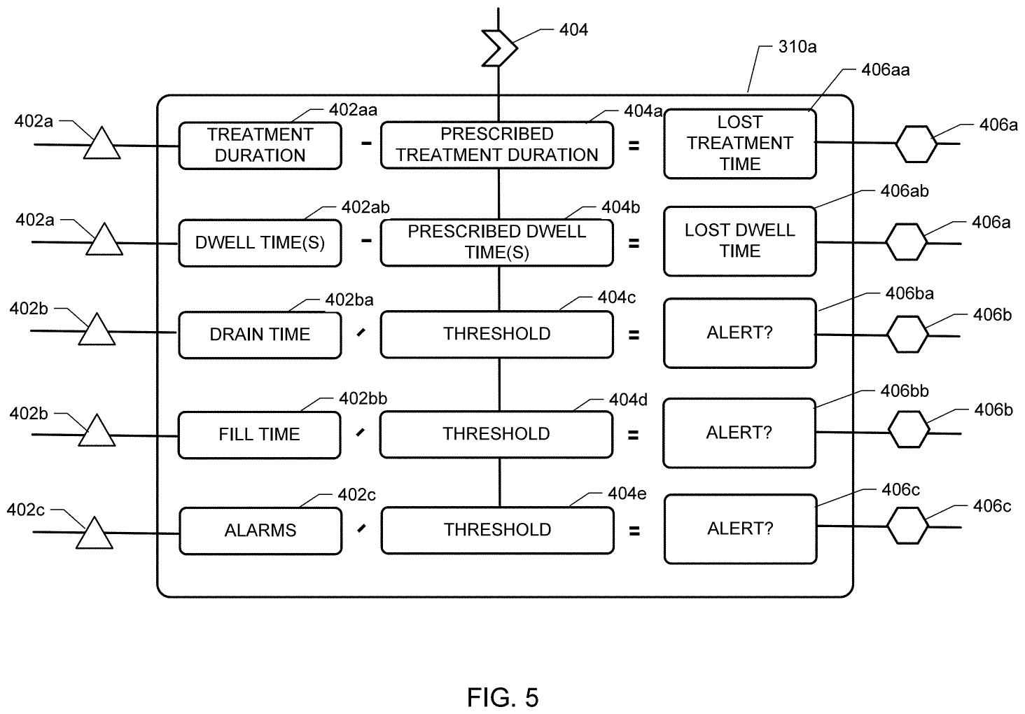

FIG. 5 is a diagram that is illustrative of at least some of the computations performed by the analytics processor 310a of FIG. 4, according to an example embodiment of the present disclosure. In the example embodiment, the analytics processor 310a is configured to compare the treatment data 402 to one or more parameters 404 that are specified in a prescribed therapy or program. At least some of the parameters 404 may include general or patient specific thresholds or limits. In the illustrated embodiment, the analytics processor 310a is configured to identify certain parameters from a prescribed therapy or program that are stored in the database 306 for the patient. The analytics processor 310a may use data labels, field names, metadata, and/or text placement to identify the parameters 404. Further, in some embodiments, patient-specific or general threshold or limits may be stored in the clinician database 306 as parameters 404.

As shown in FIG. 5, the treatment data 402a may include a treatment duration parameter 402aa, which is indicative of a treatment duration during which the therapy machine 90 administered a prescribed therapy. The analytics processor 310a is configured to compare or determine a difference between the treatment duration parameter 402aa and a prescribed treatment duration parameter 404a, which is indicative of how long the therapy machine 90 is to administer a treatment. A result of the comparison is stored as adherence analytics data 406a (i.e., a lost treatment time parameter 406aa). The lost treatment time parameter 406aa is indicative of how much treatment time was lost due to the treatment under analysis being terminated prematurely.

As shown in FIG. 4, the analytics processor 310a is configured to store the lost treatment time parameter 406aa to the clinician database 306 in a record related to the patient. The value in the lost treatment time parameter 406aa may be summed with values from previous treatments to determine a cumulative lost treatment time parameter value. In some embodiments, the analytics processor 310a may determine a ratio of the cumulative lost treatment time to total prescribed treatment time (for the treatments that were already administered) to determine a percentage of how much treatment time was lost due to the early termination of treatments.

Returning to FIG. 5, the treatment data 402a may include a dwell time parameter 402ab, which is indicative of how long the therapy machine 90 permitted a dialysis fluid to remain in a patient's peritoneal cavity before draining. The dwell time parameter 402ab may be an average for all cycles of a treatment or include dwell time values for each cycle. The analytics processor 310a is configured to compare or determine a difference between the dwell time parameter 402ab and an estimated dwell time parameter 404b, which is indicative of a estimated dwell time (e.g., based on expected or prescribed fill and drain times). A result of the comparison is stored as adherence analytics data 406a (i.e., a lost dwell time parameter 406ab). The lost dwell time parameter 406ab is indicative of how much time for absorbing UF was lost due to the treatment under analysis (or specific dwell times) being terminated prematurely.

As shown in FIG. 4, the analytics processor 310a is configured to store the dwell time parameter 406ab to the clinician database 306 in a record related to the patient. The value(s) in the dwell time parameter 406ab may be summed or combined with values from previous treatments to determine a cumulative lost dwell time parameter value. In some embodiments, the analytics processor 310a may determine a ratio of the cumulative lost dwell time to a total estimated dwell time (for the treatments that were already administered) to determine a percentage of how much dwell time was lost due to the treatments (or individual dwells) being terminated early.

Returning to FIG. 5, the treatment data 402b may include a drain time parameter 402ba, which is indicative of a length of time elapsed for the therapy machine 90 to drain the UF and dialysis fluid from the patient's peritoneal cavity. The drain time parameter 402ba may include a value for each cycle or an average over all cycles during a treatment. The analytics processor 310a is configured to compare or determine a difference between the drain time parameter 402ba and one or more thresholds 404c. A drain time value that is greater than a first threshold is indicative that it takes additional time than expected to drain dialysis fluid and UF from a patient, which may be due to a partial blockage in the catheter. A drain time value that is less than a second threshold may be indicative that less time than expected is needed to drain dialysis fluid and UF from a patient, which may be due to a misplaced catheter that causes some fluid to leak, in some instances. A result of the comparison is stored as catheter analytics data 406b (i.e., a difference parameter 406ba). If an alert is to be generated, the analytics processor 310a is configured to cause an alert to be transmitted to the clinician device 152.

As shown in FIG. 4, the analytics processor 310a is configured to store the catheter analytics data 406b to the clinician database 306 in a record related to the patient. The value(s) and/or alarm indications in the catheter analytics data 406b may be combined with values and/or indications of alarms from previous treatments to determine a cumulative drain time trend. In some embodiments, the analytics processor 310a may determine an average of drain times or an average of recent drain times for 7, 30, 50, 90 and/or 180 day averages for display in a user interface.

Returning to FIG. 5, the treatment data 402b may include a fill time parameter 402bb, which is indicative of a length of time elapsed for the therapy machine 90 to fill the patient's peritoneal cavity with dialysis fluid. The fill time parameter 402bb may include a value for each cycle or an average over all cycles during a treatment. The analytics processor 310a is configured to compare or determine a difference between the fill time parameter 402bb and one or more thresholds 404d. A fill time value that is less than a first threshold is indicative that it takes less time than expected to fill a patient's peritoneal cavity with fluid, which may be due to a misplaced catheter, in some embodiments. A fill time value that is greater than a second threshold is indicative that more time than expected is needed to fill a patient's peritoneal cavity with fluid, which may be due to a partial blockage in the catheter. A result of the comparison is stored as catheter analytics data 406b (i.e., a difference parameter 406bb). If an alert is to be generated, the analytics processor 310a is configured to cause an alert to be transmitted to the clinician device 152.

As shown in FIG. 4, the analytics processor 310a is configured to store the catheter analytics data 406b to the clinician database 306 in a record related to the patient. The value(s) and/or alarm indications in the catheter analytics data 406b may be combined with values and/or indications of alarms from previous treatments to determine a cumulative fill time trend. In some embodiments, the analytics processor 310a may determine an average of fill times or an average of recent fill times within one to two weeks for display in a user interface.

Returning to FIG. 5, the treatment data 402c may include an alarm parameter 402c, which is indicative of alarms generated during a treatment. The alarm parameter 402c may specify the types of alarms generated, a time the alarm was generated, a duration of the alarm, an indication if the condition that triggered the alarm was recovered or whether the alarm was bypassed, silenced, or escalated. The alarm parameter 402c is compared to one or more thresholds 404e. If the number of alarms generated during a treatment exceed a threshold, a patient may be experiencing alarm fatigue. Accordingly, the analytics processor 310a is configured to create an alarm parameter 406 for transmission to the clinician device 152 that is indicative of possible alarm fatigue.

As shown in FIG. 4, the analytics processor 310a is configured to store the alarm analytics data 406c to the clinician database 306 in a record related to the patient. The value(s) and/or alarm indications in the alarm analytics data 406c may be combined with values and/or indications of alarms from previous treatments to determine a total number of alarms or average number of alarms generated per treatment. In some embodiments, the analytics processor 310a may determine an average number of alarms generated when the patient uses an at-home machine 90 versus a machine 90 located in a clinic.

It should be appreciated that the analytics processor 310a may analyze and/or compare other treatment data parameters. For example, the analytics processor 310a may compare estimated UF removed during a treatment to a prescribed amount of UF to be removed to determine how closely a patient is aligned with a therapy target. The analytics processor 310a may compare actual dialysis fluid administered to the patient during a treatment to a prescribed amount. The analytics processor 310a may further compare a dextrose level to a prescribed dextrose level to ensure the patient is using the prescribed dialysis fluid. Further, the analytics processor 310a may track how a patient's blood pressure and/or weight have changed over time to detect potential fluid accumulation. In these instances, the analytics processor 310a may compare the blood pressure and/or weight to a baseline patient blood pressure or weight and/or to rate change thresholds.

FIG. 4 illustrates that in some embodiments, the application 310 of the clinician server 304 includes an interface 310b that is communicatively coupled to the analytics processor 310a and the clinician database 306. In other embodiments, the interface 310b may be included with the web portal 150. The example interface 310b is configured to read or otherwise obtain treatment data 402, analytics data 406, and/or patient data 412. The interface 310b may include one or more APIs for retrieving the data 402, 404, 406 and/or 412 from the clinician database 306.

The example interface 310b is configured to receive a request message from the application 320 on the personal mobile communication device 122 and/or the clinician device 152. The request message may identify, for example, a user interface, data fields, and/or a webpage for display. The request message may also identify a session and/or patient. In response to the request message, the interface 310b creates a response document 420 that includes the requested data. To create the document 420, the interface 310b accesses the requested patient record in the clinician database 306 and identifies the requested data 402, 404, 406 and/or 412. The data may include singular values or trends overtime that correspond to graphs displayed by a user interface. The interface 310b organizes the retrieved data into the response document 420 by data field for population by the application 320 into the appropriate user interface. The interface 310b may also label or provide a metadata identifier for the data to enable the application 320 to store the data to the appropriate data field or variable. The response document 320 may also include webpage code (e.g., http code or javascript code) that defines how the requested data is to be displayed in a web browser application 320. The interface 310b transmits the response document 420 via the web portal 150 to the personal mobile communication device 122 and/or the clinician device 152.

In some embodiments, the analytics processor 310a may transmit newly processed analytics data 406 to the interface 310b. In response, the interface 310b may update in near real time a user interface at the personal mobile communication device 122 and/or the clinician device 152. In alternative embodiments, the analytics processor 310a transmits all analytics data 406 to the clinician database 306 instead of the interface 310b. In these alternative embodiments, the interface 310b accesses the analytics data 406 from the clinician database 306 at periodic intervals (e.g., 2 seconds, 5 seconds, 10 seconds, 30 seconds, 1 minute, 5 minutes, 10 minutes, etc.) to update the user interfaces on the personal mobile communication device 122 and/or the clinician device 152.

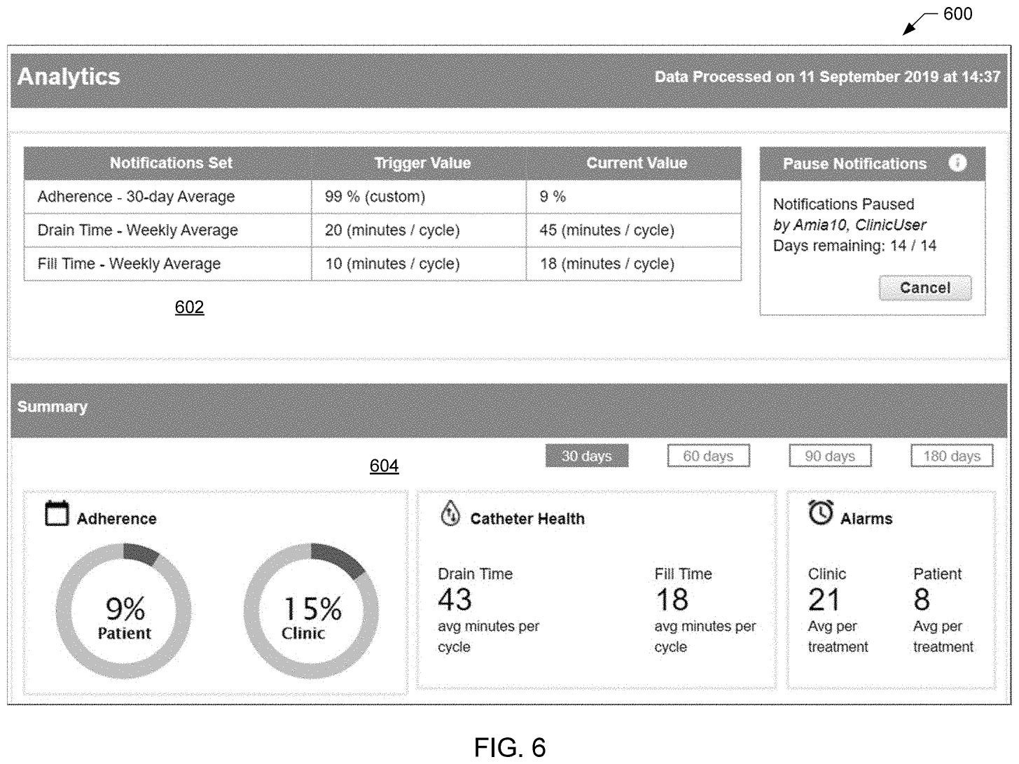

FIGS. 6 to 13 illustrate example user interfaces that are created, rendered, or otherwise provided by the application 320 on the personal mobile communication device 122 and/or the clinician device 152 using at least some data 402, 404, 406, and/or 412 from the clinician database 306, according to an example embodiment of the present disclosure. FIG. 6 shows a diagram of an example dashboard user interface 600, according to an example embodiment of the present disclosure. The dashboard user interface 600 provides a holistic picture of a specified patient's adherence to one or more prescribed therapies or programs that are administered by one or more therapy machines 90 (e.g., medical fluid delivery machines). The user interface 600 may be displayed by the application 320 on the clinician device 152 after selection, for example, of a patient's name among a list of patients that the clinician is treating or otherwise responsible for overseeing. The user interface 600 may be displayed by the application 320 on the personal mobile communication device 122 as a front or loading page since the patient will not be permitted to see information of other patients.

The user interface 600 includes a trigger section 602. In the illustrated example, the trigger section 602 displays a current value of adherence analytic data and a trigger or threshold value for generating an alert. The trigger section 602 also displays current values of a weekly average for drain time and fill time treatment data in addition to trigger or threshold values. The user interface 600 also includes a summary section 604, which shows adherence analytic data for a patient compared to a clinic average (or an average of a plurality of patients in multiple clinics). In the illustrated example, the patient has a lower adherence compared to a population of patients that receive treatment in clinics (from which treatment data is received). The summary section 604 also shows catheter treatment data as specified by average fill and drain times. The summary section 604 further shows an average number of alarms per treatment for a patient compared to a clinic average. The summary section 604 enables a user to view the data for the past 30 days, 60 days, 90, days, and/or 180 days.

In some instances, the application 320 is configured to enable a clinician to specify the trigger or threshold values for adherence, catheter health, and/or alarms. For instance, selection of a link or field in the user interface 600 may cause the application 600 to load another user interface that enables trigger values and/or thresholds to be modified by the clinician. The thresholds may include a discrete number or a percent increase over a time period or between treatments. Setting a trigger value or threshold causes the value to be transmitted to the interface 310b of the clinician server 304, and stored to the clinician database 306 as a threshold parameter 404. Setting a trigger value causes, for example, the interface 310b or the analytics processor 310a to display an icon, notification, or other alert on a patient or treatment dashboard user interface (or via a push notification message) that is indicative of the treatment data or analytic data exceeding the threshold or trigger.

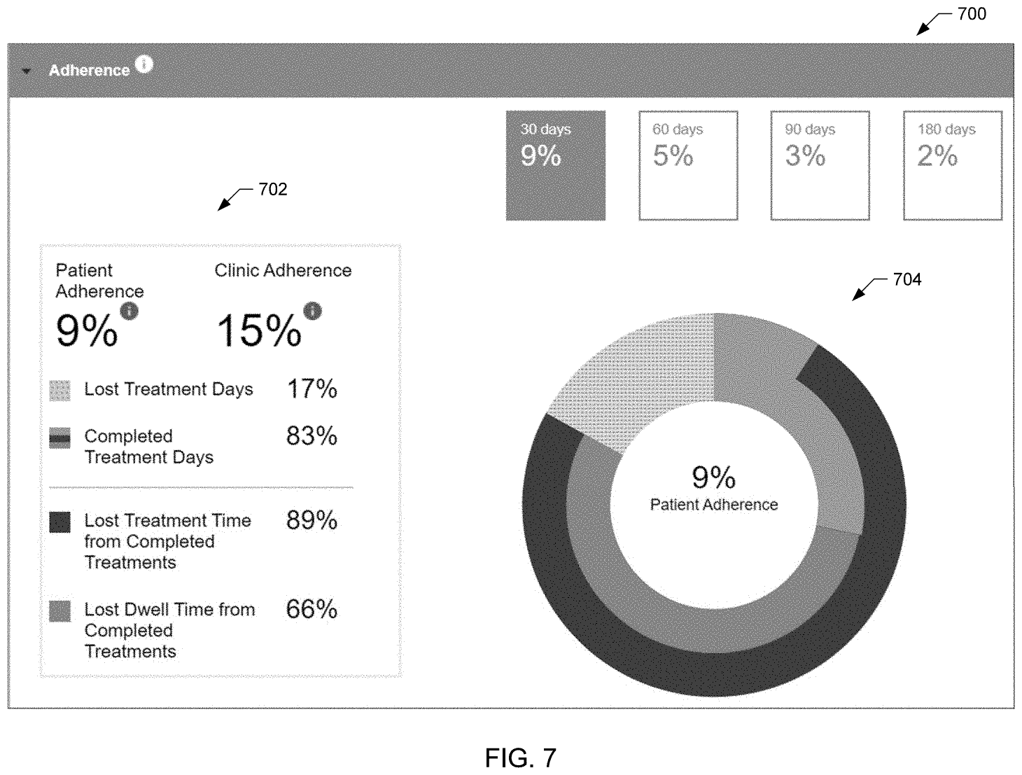

FIG. 7 shows an adherence user interface 700 that displays adherence analytic data, according to an example embodiment of the present disclosure. The adherence user interface 700 is indicative as to whether a particular patient meets a prescribed treatment days, total treatment time, and/or estimated dwell time. Such information provides for early intervention by a clinician by providing for early detection of a decline in treatment administration. Generally, less than 90% adherence is associated with a significant increase in technique failure, peritonitis rates, hospitalization, and/or rate of death. The user interface 700 may be displayed by the application 320 upon a user selection of the adherence analytic data in the user interface 600.

The user interface 700 includes a numerical section 702 and a graphical section 704 that shows adherence analytic data over a 30-day, 60-day, 90-day, or 180-day period. The interface 700 includes a summary of adherence over the different time periods to provide a patient adherence trend. The sections 702 and 704 show patient adherence compared to average adherence for patients of the same clinic or a plurality of patients in one or more clinics (from which data is received). Further, the sections 702 and 704 show a percentage or ratio of lost treatment days in which a treatment was not performed as scheduled, a percentage or ratio of completed treatment days in which a scheduled treatment was performed, a percentage or ratio of lost treatment time from completed treatments (indicative of a patient ending a treatment prematurely), and a percentage or ratio of lost dwell time from completed treatments (indicative of a patient bypassing full dwell phases of a cycle). The lost dwell time information is especially important since it conveys how much opportunity or prescribed time was lost for absorbing a patient's UF for removal.

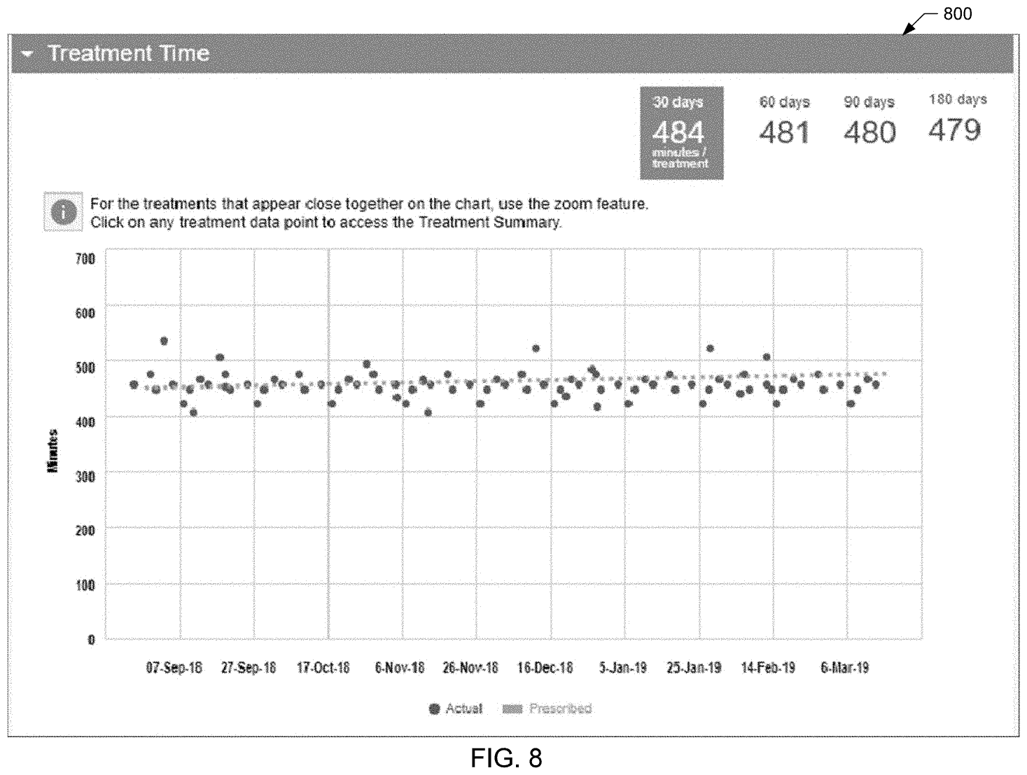

FIG. 8 shows a treatment time user interface 800 that may be displayed by the application 320 upon selection of treatment data in the user interfaces 600 or 700. The user interface 800 shows a scatter plot of treatment time for a patient relating prescribed treatment time to actual treatment time. In the illustrated example, the interface 310b of FIG. 4 is configured to transmit, within a document 420, the individual treatment times and prescribed treatment times for each day of scheduled treatment. The application 320 writes the treatment data from the document 420 into the data fields associated with the user interface 800 to generate the scatter plot. As shown, the prescribed treatment time increases slightly over time from about 450 minutes to 480 minutes. In comparison, the actual treatment time varies, generally being less than the prescribed treatment time. A user may hover over each data point on the scatter plot of the user interface 800 to cause the application 320 to display a time, date, and/or prescribed/actual treatment time values. The user interface 800 also shows average actual treatment times over a 30-day, 60-day, 90-day, or 180-day period.

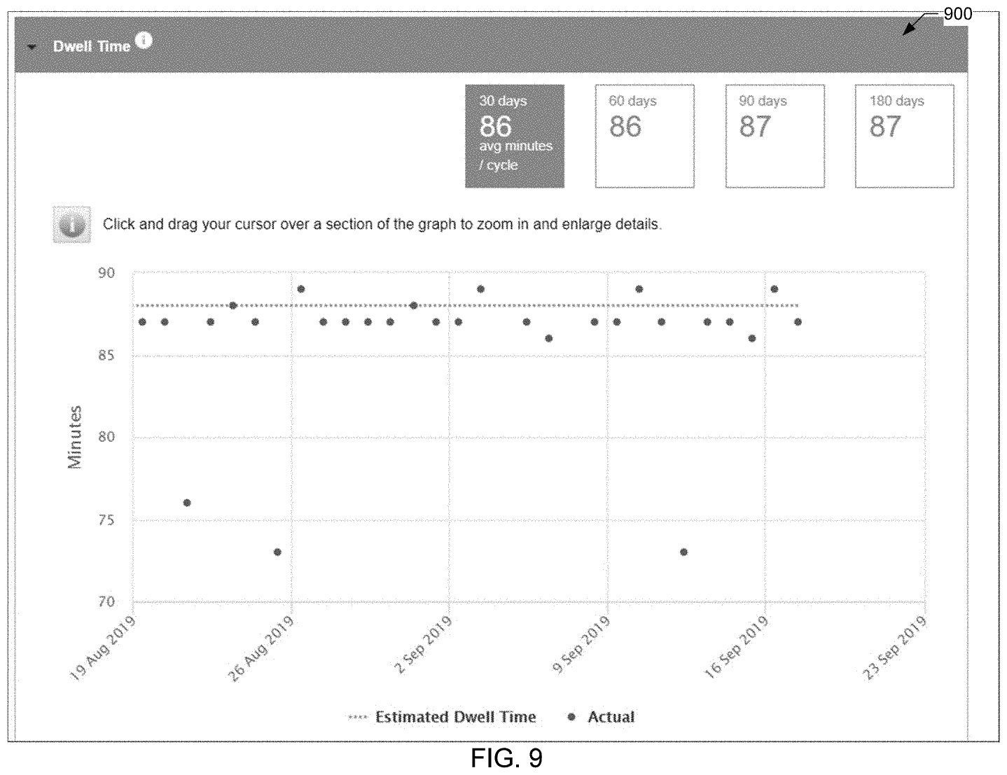

FIG. 9 shows a dwell time user interface 900 that may be displayed by the application 320 upon selection of dwell data in the user interfaces 600 or 700. The user interface 900 shows a scatter plot of dwell times for a patient that relates estimated dwell times (calculated from prescribed or specified fill and drain times) to actual dwell times. As shown, the estimated dwell times remain constant at about 87 minutes per cycle in a treatment. In comparison, the actual dwell times vary, generally being less than the estimated dwell time. A user may hover over each data point on the scatter plot to cause the application to display a time, date, and/or estimated/actual dwell time values. The user interface 900 also shows average actual dwell times over a 30-day, 60-day, 90-day, or 180-day period.

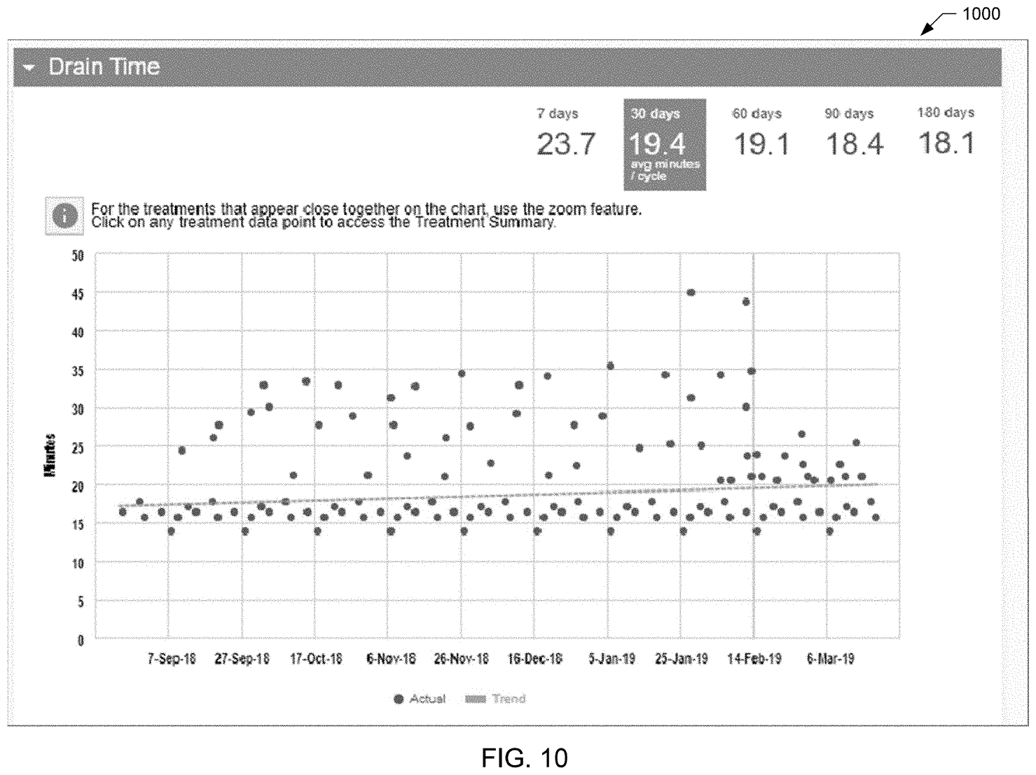

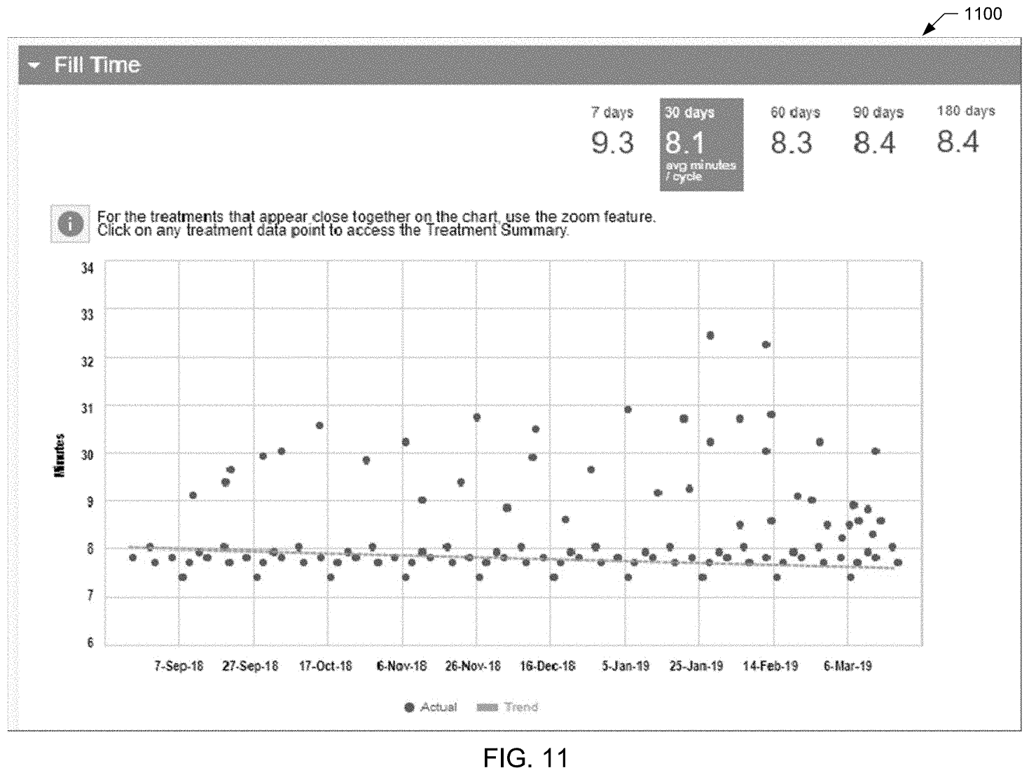

FIGS. 10 and 11 show respective drain and fill time user interfaces 1000 and 1100 that may be displayed by the application 320 upon selection of drain or fill time analytic data in the user interfaces 600 or 700. The drain and fill time user interfaces 1000 and 1100 show scatter plots of average drain and fill times per treatment. The displayed information provides an early indication of an issue with a patient's catheter, where a slow decrease or increase in fill or drain times may be unnoticeable when viewed on a day-to-day basis. The data may be indicative of a catheter flow restriction, misalignment, constipation, and/or an accumulation of fibrin strands.

The drain time user interface 1000 shows individual average drain times per treatment in relation to a trend line, which shows in this embodiment that the average drain time has increased from 17 minutes to 19 minutes. The user interface 1000 also shows average actual drain times over a 7-day, 30-day, 60-day, 90-day, or 180-day period, indicating that a recent increase in drain time may be indicative of an issue with the catheter. The fill time user interface 1100 shows individual average fill times per treatment in relation to a trend line, which shows that the average fill time in this embodiment has decreased from 8 minutes to 7.5 minutes. The difference between the change of the fill times relative to the change of drain times may provide information indicative of a flow restriction, where a faster fill rate may not be as affected by a partial blockage of the catheter. A user may hover over any of the data points in the user interfaces 1000 and 1100 to view the date, time, and value/minutes of the value.

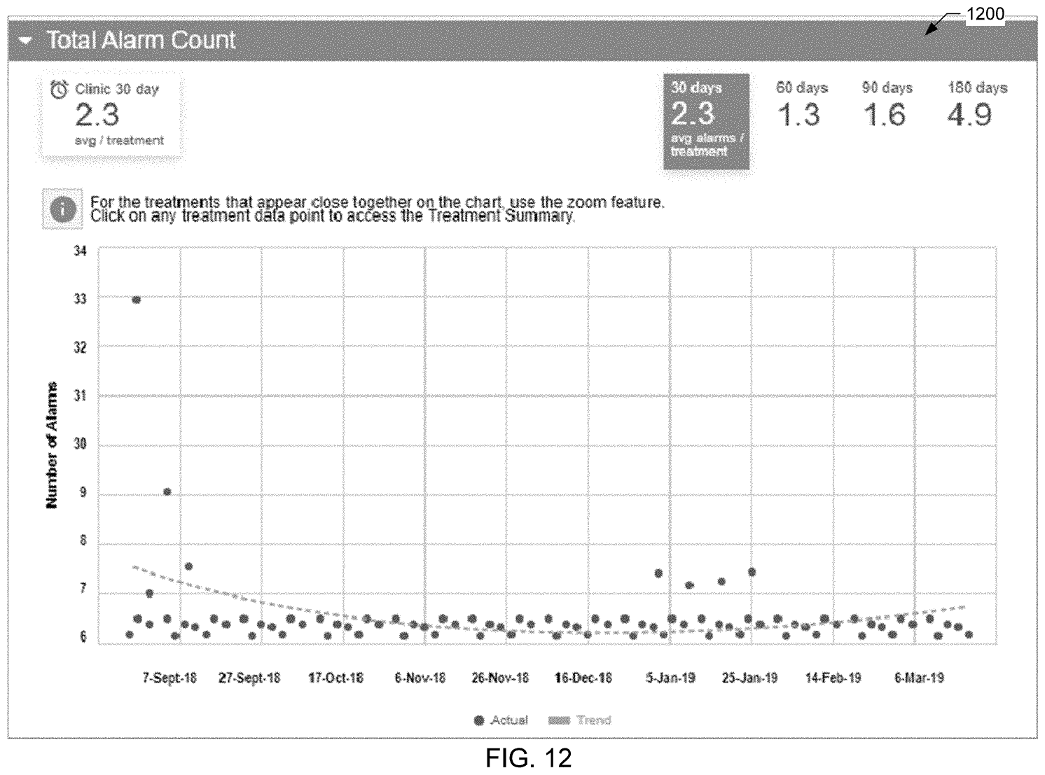

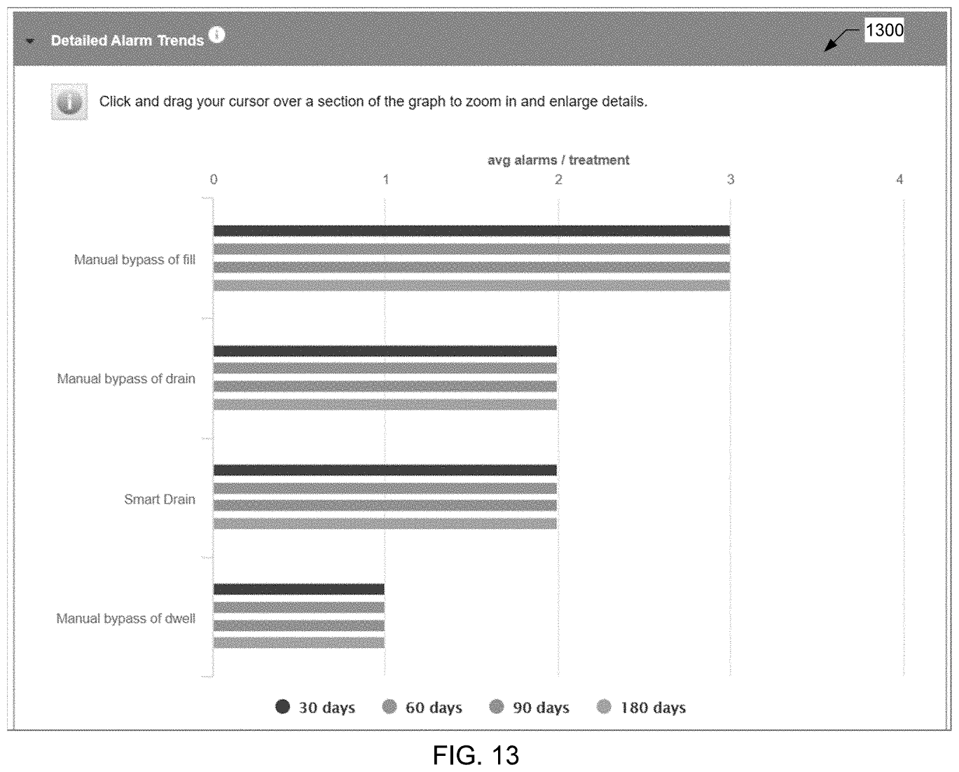

FIGS. 12 and 13 show alarm user interfaces 1200 and 1300 for displaying alarm analytic data, according to example embodiments of the present disclosure. The alarm user interface 1200 of FIG. 12 includes a scatter plot of an average number alarms generated by a therapy machine 90 during a treatment. The plot also includes a trend line of the alarms. The plot may be used by clinicians for early intervention to resolve alarm issues with patients to provide better rest periods and better adherence to their prescribed therapy or program. An increase in alarms per treatment may be indicative of adherence issues, catheter issues, and/or technique failure. An increase in alarms can lead to patient sleep disturbances and alarm fatigue. In some instances, a user interface may show dates/times the alarms were generated as well as types of alarms and/or indications as to whether the alarms were silenced, escalated, or resolved. The user interface 1200 also shows an average number of alarms over a 30-day, 60-day, 90-day, or 180-day period.

FIG. 13 shows a user interface 1300 that includes average alarm types per treatment over different time periods. The alarm types include, for example, a manual bypass of a fill, a manual bypass of a drain, a smart drain alert, and a manual bypass of a dwell time. The illustrated alarms are indicative that a patient is ending fill, dwell, and drain phases earlier than prescribed. In other embodiments, the user interface 1300 may display alarms indicative of access disconnection detections, line occlusions, etc. In some embodiments, the alarms may also indicate if measured treatment data exceeds one or more thresholds for blood pressure pre/post treatment, blood glucose, pre/post patient weight, heart rate, drained UF, kt/V, CCI, fill volume, drain volume, transporter type, number of manual exchanges, etc. The number of alarms and types of alarms shown in the user interface 1300 may be used by a clinician for therapy intervention to help reduce the number of reoccurring alarms or identify patient treatment adherence issues.

### B. Treatment Recommendation Embodiment

Returning to FIG. 4, the example application 310 on the clinician server 304 may include a guidance processor 310c configured to provide evidence-based analytics or apply evidence-based models. The guidance processor 310c is configured to analyze the data 402, 404, 406, and/or 412 to provide recommendations and/or guidance regarding clinical benefits and risks for a prescribed therapy and/or program. The recommendations and/or guidance is actionable by a clinician for actively working with a patient to improve adherence, catheter issues, and/or alarm issues.

In some embodiments, the clinician database 306 stores a data structure 428 that relates recommendations and/or guidance to portions or ranges of at least some of the data 402, 404, 406, and/or 412. The recommendations and/or guidance may be provided by a nationally recognized authority, such as the ISPD, the NKF, the NKF DOQI, and/or peer-reviewed publications. The recommendation or guidance may include recommended therapy or program settings or parameters to modify a prescribed therapy or program. The recommendations or guidance may also include recommended settings or ranges for notification thresholds. The recommendations or guidance may further include definitions of analytics/metrics, links to websites with additional information, typical clinician values for similar patients, potential patient-related risks, and/or actions for a clinician to undertake, such as contacting a patient, scheduling maintenance for a catheter or therapy machine 90, or silencing some alarms on the therapy machine 90.

The example guidance processor 310c of FIG. 4 is configured to compare the data 402, 404, 406, and/or 412 to one or more thresholds or ranges for the recommendations and/or guidance provided in the data structure 428. If at least some of the data 402, 404, 406, and/or 412 corresponds to a recommendation or guidance, the guidance processor 310c creates a recommendation document 430 that includes the identified recommendations or guidance. The guidance processor 310c causes the interface 310b to transmit the recommendation document 430 to the application 320 for display.

To create the guidance document 430, the guidance processor 310c may include text of the related recommendation and/or guidance. Additionally or alternatively, the guidance processor 310c may access a list of pre-specified guidance or recommendations that are stored within the data structure 428. The guidance processor 310c identifies the pre-specified guidance or recommendation that corresponds to the comparison. After making the identification, the guidance processor 310c writes the pre-specified guidance or recommendation to the recommendation document 430. In some embodiments, the guidance processor 310c may access a webpage or other web-based content via one or more links within related guidance or recommendations to obtain text or multimedia content for population into the guidance document 430.

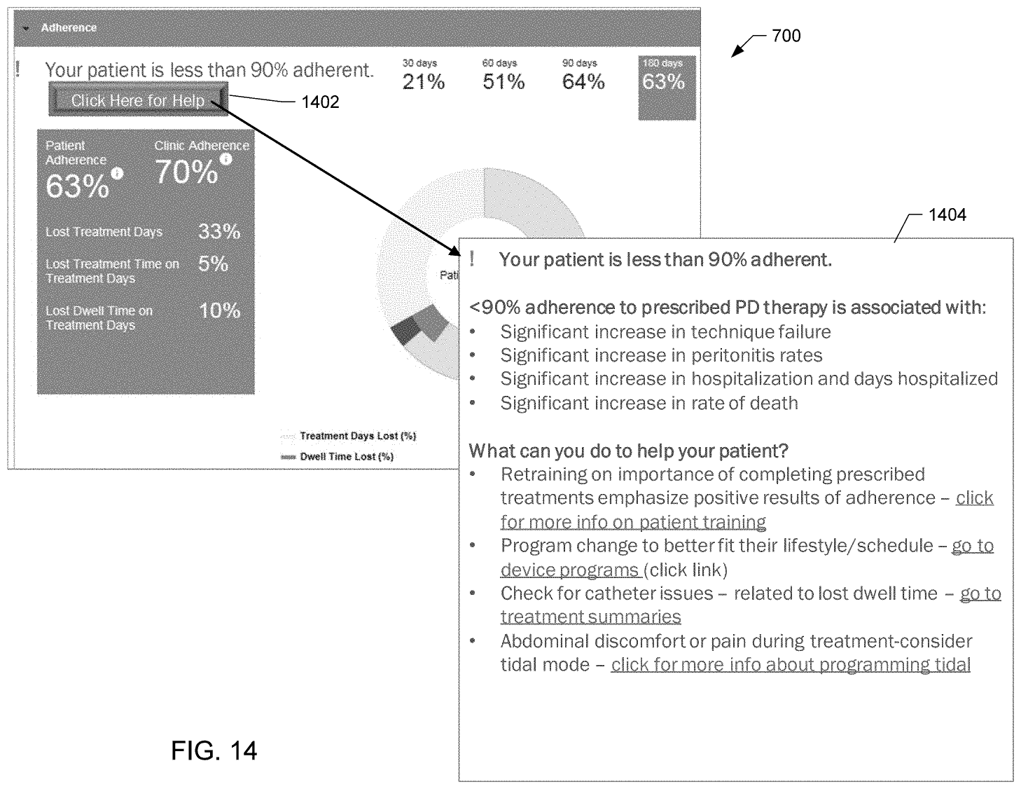

FIG. 14 shows the adherence user interface 700 of FIG. 7 with an icon 1402 that enables a user to view recommendations or guidance determined by the guidance processor 310c. The application 320 may display the icon 1402 after the recommendation document 430 is received. Alternatively, selection of the icon 1402 may cause an instruction to be transmitted to the guidance processor 310c to cause the recommendation document 430 to be created.

In some embodiments, selection of the icon 1402 causes the application 320 to display the recommendation user interface 1404. In the illustrated example, the guidance processor 310c concurrently or previously determined that recommendations exist for patients that have an adherence analytics value that is less than 90%. The guidance processor 310c extracts the associated recommendations and guidance, which are shown in the user interface 1404. The guidance includes a brief explanation of possible causes for the low adherence rate in addition to actions the clinician can take to help the patient improve adherence. The recommendations in the user interface 1404 include links to websites or webpages with additional information or content to assist the clinician. In some instances, the links may cause an email program to open, cause a text messaging program to open, or initiate a call to the patient to enable the clinician to quickly connect to the patient. In other instances, the links may cause a user interface to open with editable fields for a prescribed therapy or program. A clinician may use the interface to modify or create a new prescribed therapy or program if warranted, which is transmitted to the clinician server 304 for processing/validation prior to transmission to the therapy machine 90.

### C. Treatment Analytics Example Procedure

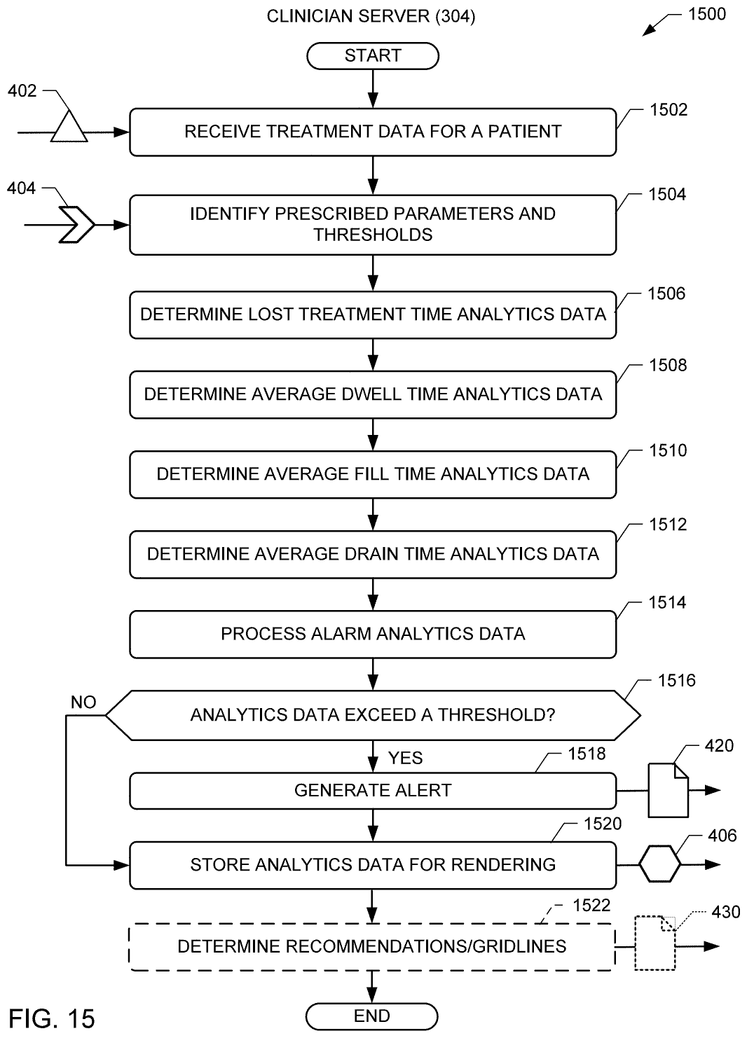

FIG. 15 is a flow diagram of an example procedure 1500 to create analytics data 406 using treatment data 402 and/or thresholds/parameters 406 of a prescribed therapy or program, according to an example embodiment of the present disclosure. Although the procedure 1500 is described with reference to the flow diagram illustrated in FIG. 15, it should be appreciated that many other methods of performing the steps associated with the procedure 1500 may be used. For example, the order of many of the blocks may be changed, certain blocks may be combined with other blocks, and many of the blocks described may be optional. In an embodiment, the number of blocks may be changed based on which analytic data is determined. Further, the step of determining recommendations and/or guidelines may be omitted. The actions described in the procedure 1500 are specified by one or more instructions, and may be performed among multiple devices including, for example, the clinician server 304 and/or the clinician database 306.

The example procedure 1500 begins in FIG. 15 when the clinician server 304 receives treatment data 402 for a patient from one or more therapy machines 90 (e.g., medical fluid delivery machines) (block 1502). The clinician server 304 receives the treatment data 402 after a treatment has finished. In other instances, the clinician server 304 receives the treatment data on a periodic basis, such as every 5 seconds, 10 seconds, 30 seconds, 1 minute, 5 minutes, 15 minutes, 1 hours, 24 hours, etc. In some embodiments, the clinician server 304 may convert the received treatment data 402 from an HL7 format into a JSON format or text-based format. The clinician server 304 identifies parameters of prescribed therapies or specified thresholds 404 that correspond to the received data (block 1504). For example, the clinician server 304 may access a patient record in the clinician database 306 to determine parameters of a prescribed therapy. The clinician server 304 may also access the clinician database 306 to determine any default or clinician-specified thresholds or triggers for generating alerts.

The example clinician server 304 then determines analytic parameters by comparing or determining differences between at least some of the treatment data 402 and the parameters of prescribed therapies and/or specified thresholds 404. This may include determining lost treatment time analytic data 406aa (block 1506), average dwell time analytic data 406ab (block 1508), average fill time analytic data 406bb (block 1510), and average drain time analytic data 406ba (block 1512), as described above in connection with FIG. 5. The example clinician server 304 also processes the treatment data 402 to determine alarm analytic data 406c by determining a number of alarms generated, types of the alarms, and/or whether the alarms were bypassed, silenced, resolved, or escalated (block 1514).

The clinician server 304 then determines if any of the analytics data 406 exceeds one or more thresholds (block 1516). If a threshold is exceeded, the clinician server 304 generates an alert, which is transmitted to an application 320 on a clinician device 152 via a document 420 (block 1518). In other instances, the alert is provided by the clinician server 304 after detecting the application 320 is active and/or connected for receiving the document 420. The alert is indicative that a clinician-specified or default threshold has been exceeded, such as an adherence rate, an average dwell time, an average drain time, an average fill time, a number of alarms generated, etc. The example clinician server 304 also stores the analytics data 406 to one or more patient records in the clinician database 306 (block 1520). The clinician server 304 or the web portal 150 may later retrieve the stored data 406 upon request for rendering and display on an application 320 at a personal mobile communication device 122 or the clinician device 152.

The clinician server 304, in some embodiments, may determine if a recommendation or guideline is to be provided based, for example, on whether an alert was generated or based on the analytics data 406. If a recommendation is to be generated, the clinician server 304 determines the appropriate or corresponding recommendation, and creates a recommendation document 430 for transmission and display via the application 320 (block 1522). The example procedure 1500 of FIG. 15 may then end. The procedure 1500 may start again when additional treatment data is received.

### III. TREATMENT PREDICTIVE EMBODIMENTS

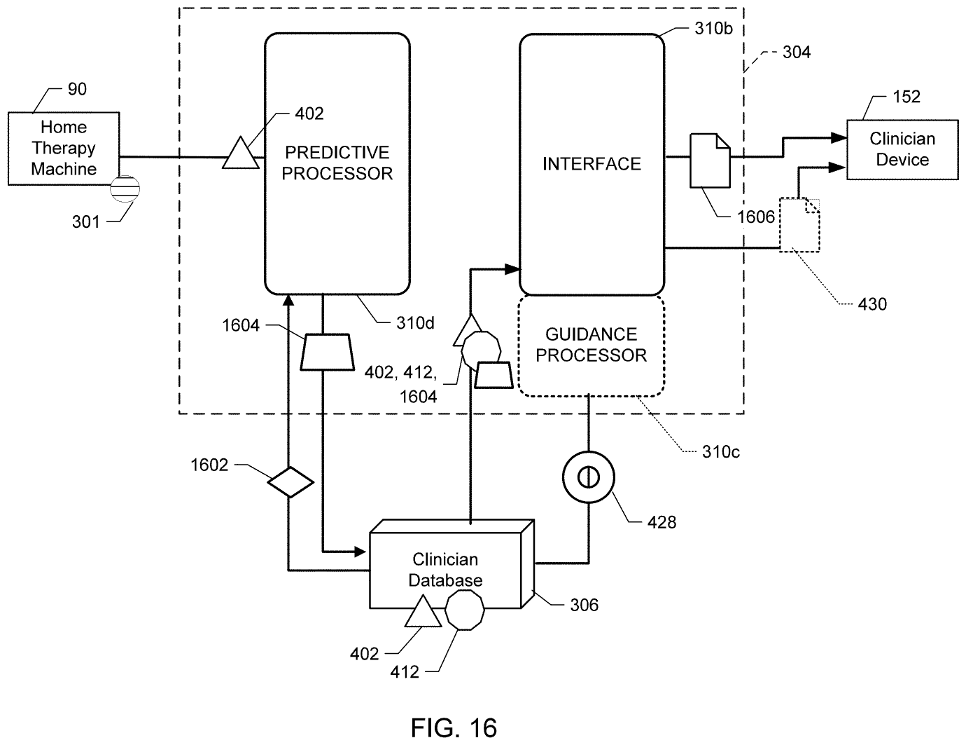

In some embodiments, the example clinician server 304 includes an application 310 that is configured to use one or more AI models, machine learning models, engines, and/or predictive analytics to determine a likelihood that a patient may stop or reduce treatments performed by the therapy machine 90. FIG. 16 is a diagram of the application 310 of the clinician server 304 including a predictive processor 310d, according to an example embodiment of the present disclosure.

The example predictive processor 310d may include one or more AI models, such as a LightGBM model and/or a Feedforward neural network model, that is configured or trained to predict patient stoppage at least five to seven days in advance, and up to 21 to 30 days in advance. The predictive processor 310d is trained using a training data set 1602 that comprises treatment data 402 and/or logs for patients on a rolling six to twelve month period. The treatment data 402 is analyzed during this time period to identify patients that experienced gaps in treatment of at least five to 14 days. The predictive processor 310d correlates the treatment data 402, the patient data 412, and/or any thresholds stored in the records of the clinician database 306 to the identified patients. As such, the predictive processor 310d possesses a data set that identifies patients that stopped treatment, and the treatment and personal characteristics of those patients that stopped treatment versus patients that did not stop treatment.

The example predictive processor 310d is configured to determine a model output 1604 by comparing the treatment data 402 and/or patient data 412 to the modeled patients. The model output 1604 includes an average predicted probability of the patient under consideration stopping or reducing their treatments of a prescribed therapy or program. The predicted probability is determined by comparing the treatment data 402 (including a history of the patient's treatment data 402) and the patient data 412 to the training data or modeled data to determine how closely the data of the patient matches or correlates the training or modeled data set using at least three to five (or more) parameters. For instance, treatment data 402 of a patient that is almost identical to treatment data of other patients that have stopped treatments is determined by the predictive processor 310d to have between a 95% to 100% predicted probability of stopping treatment. However, if the treatment data of the patient under consideration has deviations from the data of other patients that have stopped treatments (meaning the data begins to resemble data of patients that continued treatments), the predicted probability determined by the predictive processor 310d is lower.

In some embodiments, the training data set 1602 includes a table of treatment data 402 and/or patient data 412, with each row specifying a patient-day of a prescribed treatment. Columns may include the treatment data 402 and/or the patient data 412 for that patient-day treatment (e.g., input features). A column may also include an indication as to whether the patient stopped treatments afterwards (e.g., a target variable). Further, at least some of the training data set 1602 may be excluded from the modeling process and instead used as holdout or verification data to ensure the training data properly trained the predictive model.

In some embodiments, after training, the predictive model is configured to use recursive feature elimination to remove parameters that do not materially contribute to determination of a concern score. Additionally or alternatively, parameters of the predictive model may be optimized using a Bayesian search over a parameter space. Further, the predictive model may be tested for stability before use to ensure one or more predictions performed on training models did not become unstable using treatment data and/or patient data from longer time intervals.

Using the trained predictive models, the predictive processor 310d may classify the predicted probability as a ‘concern score’, and report the concern score to a related clinician if it exceeds a threshold (e.g., greater than a 50% chance of ending treatment). In addition to reporting the concern score, the predictive processor 310d may include or identify critical parameters or contributing factors that caused a relatively high score, thereby providing transparency for the AI or machine learning models or engines. In some embodiments, the critical parameters may be weighted based on importance or correlation with the concern score. Further, in some embodiments, the predictive processor 310d may determine a first concern score regarding a probability a patient will end a prescribed therapy (for at least two weeks) and a second concern score regarding a probability a patient will reduce a treatment frequency or duration.

In some instances, the predictive processor 310d is configured with a supervised learning approach, which compares current and historical treatment/patient data to an actual outcome of a patient continuing or stopping a treatment (e.g., a target variable). The predictive processor 310d determines that a patient stopped treatment by identifying when treatment data was not uploaded by a machine 90 for a period of at least two weeks beginning at some point during a prediction window (a timeframe of the prediction, such as within the next two weeks of the date of interest, starting one week from that date). In other words, the predictive processor 310d determines that no treatment exists for at least two weeks, which is indicative that the patient (in the training set) has stopped treatments. To determine a concern score of a target patient using the patients from the training set, the AI or machine learning systems of the predictive processor 310d determine an optimal fitting model to the modeled data using the parameter inputs. The predictive processor 310d may be configured to use techniques such as cross-validation and testing against a “holdout set” not used for training to avoid over-fitting the model.

In some instances, the predictive processor 310d uses a model that receives patient/treatment data inputs and provides deltas between current and prior values to show trends, and cumulative counts of values (such as alerts) over a timeframe (such as 28 days). In addition, the predictive processor 310d may include other derived data elements such as a pulse pressure (difference between systolic and diastolic blood pressure in a single measurement). For each day, the predictive processor 310d may generate a target variable concern score in addition to the trends/cumulative counts. The predictive processor 310d provides the patient treatment data inputs (a set of inputs per patient per day) to the predictive model for comparison to the target variable for that patient (e.g., 1 or 0 to indicate whether the patient did (or did not) stop using the therapy machine 90 during the prediction window). The predictive processor 310d may then use one or more machine learning models (like LGBM, Neural Networks, and Logistic Regressions) to generate a probability (a decimal between 0 and 1) that the target variable is “1” (e.g., that the patient will stop using the therapy machine 90 beginning sometime during the prediction window).

In some embodiments, only a small subset of the treatment data for a patient under consideration may be used in the analysis. For instance, the predictive processor 310d may exclude the most recent treatment data 402 (e.g., data within five to seven days) for a patient under consideration. Further, the predictive processor 310d may only use treatment data received within the last 5 to 30 days, preferably between 7 to 21 days for the analysis. Thus, a patient's past long term compliance cannot bias future predicted therapy adherence, but rather short term trends are considered, which are more indicative of near-term therapy stoppage.

The critical parameters used by the one or more patient predictive models and/or engines of the predictive processor 310d include:
drain time (weighted average, maximum, average, minimum, variation and/or cumulative drain time per treatment),
drain volume (initial or cumulative, may include at least one of a weighted average, maximum, average, minimum, and/or variation of drain values),
estimated day/night UF removed amount per treatment (includes at least one of a weighted average, maximum, average, minimum, and/or variation of estimated UF removed),
dwell time (weighted average, maximum, average, minimum, variation or cumulative drain time per treatment),
fill time (average or cumulative fill time per treatment),
escalating messages/alarms/alerts generated by the machine 90 between treatments (may include a weighted average, maximum, average, minimum and/or variation between monthly/weekly/per-treatment alarms, messages, or alerts),
cumulative alarms/alerts,
number of days experience with the therapy machine 90 (including prior prescribed therapies),
number of days of treatment at a clinic,
number of days the patient has been on the prescribed therapy,
number of times the prescribed therapy has been revised,
average hours between separate treatments (e.g., between day and night treatments),
number of cycles in each treatment,
patient age,
patient gender,
prescribed therapy identifier,
total treatment volume (may include a weighted average, maximum, average, minimum, and/or variation of treatment fluid volume),
treatment duration (may include a weighted average, maximum, average, minimum, and/or variation of treatment durations),
patient pre/post treatment weight (may include a weighted average, maximum, average, minimum, and/or variation of patient weight),
patient blood pressure (includes at least one of a weighted average, maximum, average, minimum, variation diastolic and/or systolic blood pressure),
patient heart rate, and
patient renal condition.

It should be appreciated that the predictive model uses at least three of the above-parameters, and as many as twenty to thirty parameters. If a concern score exceeds a threshold, the interface 310b is configured to create a document 1606 that includes the concern score and a top number of contributing critical parameters. The interface 310b may also include the patient's value for each critical parameter that was used in the patient predictive models/engines of the predictive processor 310d. In other embodiments, the concern score and top critical parameters may be provided by the interface 310b when requested by the clinician via the application 320.

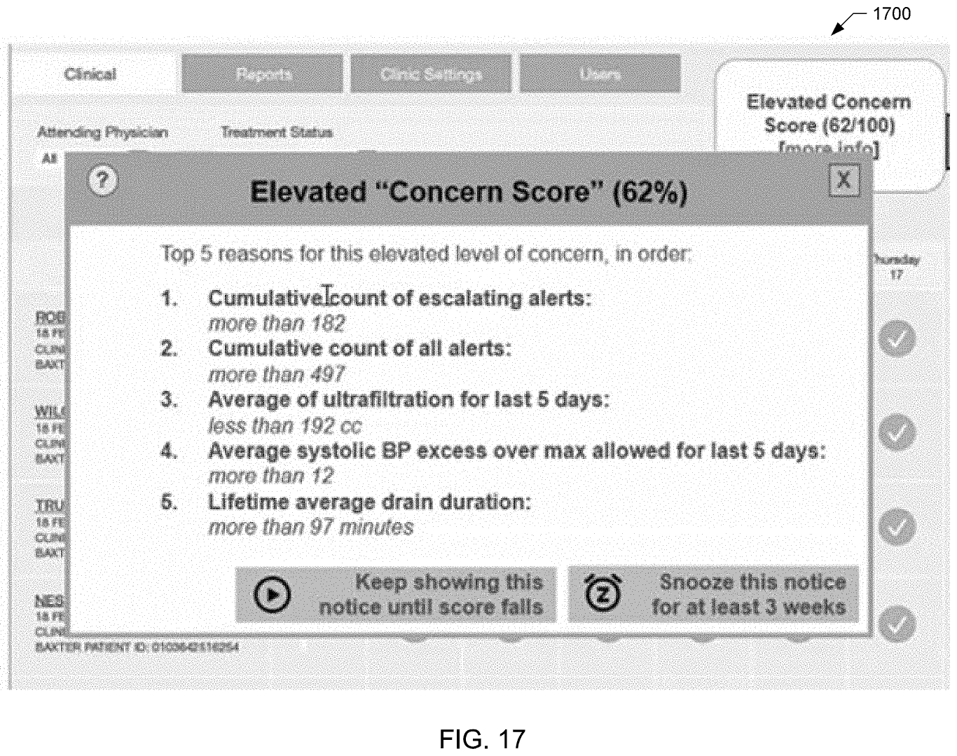

FIG. 17 shows a concern score user interface 1700, according to an example embodiment of the present disclosure. The user interface 1700 displays the concern score and a top number of critical parameters (in this example five top critical parameters). The critical parameters include the values for the patient. In this example, the patient has a 62% chance of stopping treatment in the future (e.g., in the next one to four weeks). The indicators that the patient may stop treatment include an escalating number of alerts, a cumulative count of alerts, average amount of UF removed, the patient's blood pressure, and long drain times.

A clinician reviewing this information has an opportunity to intervene prior to the patient ending treatment. The patient may convey that they are getting frustrated using the machine (as indicated by the alerts and blood pressure) while at the same time feeling that the benefits are being reduced (low UF removal) while taking longer (higher drain time). In response, the clinician can reach out to the patient to determine if a different dextrose level is needed to help remove UF more quickly. The clinician may also help the patient avoid setting off alarms and/or alerts. Altogether, the clinician has a good chance of keeping the patient on treatments by being able to anticipate and act before a patient stops the therapy

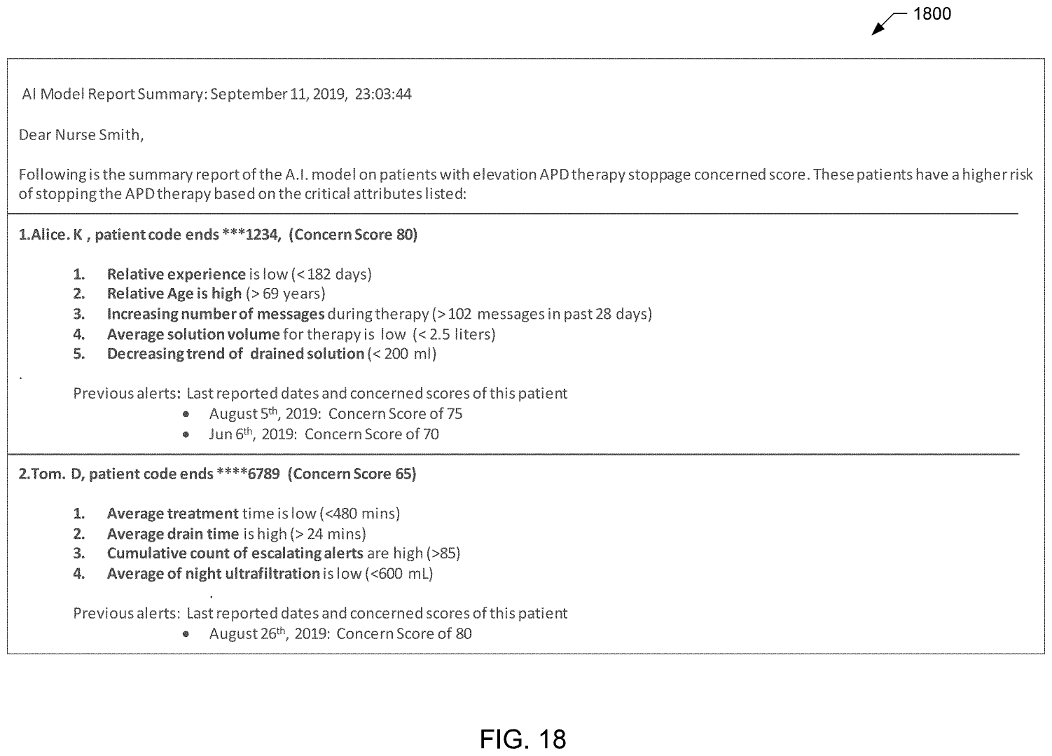

FIG. 18 shows another embodiment of a concern score user interface 1800, according to an example embodiment of the present disclosure. The user interface 1800 includes reports for patients for which a clinician is responsible for treating. The user interface 1800 includes a patient name, identifier, and concern score. The user interface 1800 also includes critical parameters for each of the patients. It should be appreciated that the critical factors differ between the patients. This patient-specific analysis enables the clinician to narrow in and address the specific causes of the patient's risk of stopping treatment. The user interface 1800 also includes a listing of previous concern scores to show how the patient is trending.

In some embodiments, the application 310 of the clinician server 304 may include the guidance processor 310c to provide recommendations for addressing the critical parameters. The guidance processor 310c is configured to compare a patient's critical parameters to values or ranges of values assigned to different recommendations and/or guidance provided in a data structure 428 (shown in FIG. 16). The recommendations or guidance may be determined for each critical parameter, the top identified critical parameters, or generally provided based on a concern score.

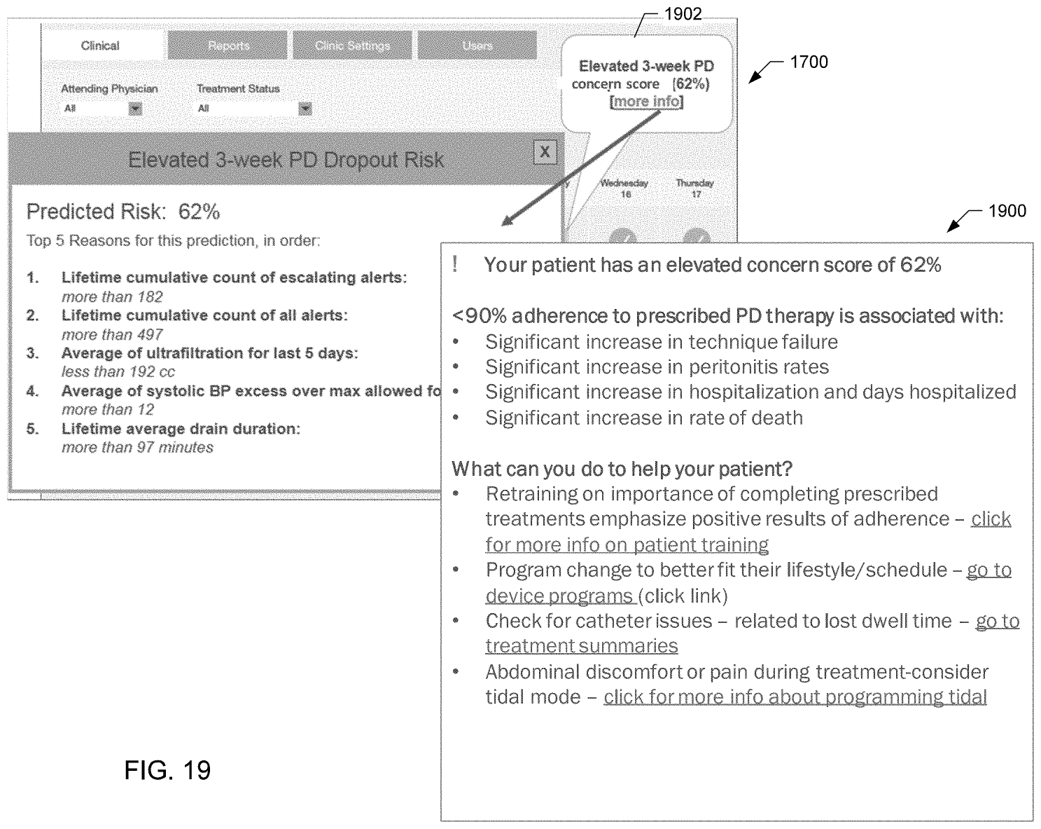

If at least some of the critical parameters and/or a concern score correspond to a specific recommendation or guidance, the guidance processor 310c creates a recommendation document 430 that includes the identified recommendations or guidance. The guidance processor 310c causes the interface 310b to transmit the recommendation document 430 to the application 320 for display. FIG. 19 shows the adherence user interface 1700 of FIG. 17 with an icon 1902 that enables a user to view recommendations or guidance determined by the guidance processor 310c. Selection of the icon 1902 causes the application 320 to display the recommendation user interface 1900.

In the illustrated example, the guidance processor 310c determines that recommendations exist a patient that has a concern score of 62%. The guidance processor 310c extracts the associated recommendations and guidance, which are shown in the user interface 1900. The guidance includes a brief explanation of possible causes for the concern score in addition to actions the clinician can take to help the patient improve adherence to a prescribed therapy. The user interface 1900 accordingly guides a clinician to improve a patient's adherence to a prescribed therapy based on predictive analytics of patients undergoing similar treatments.

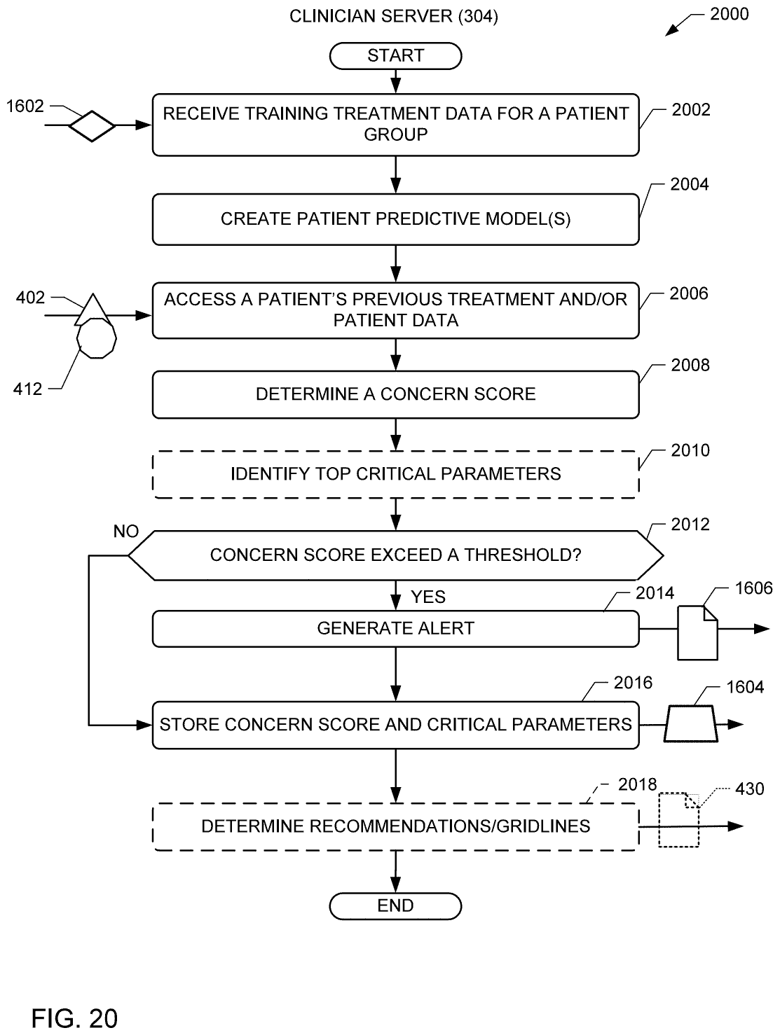

FIG. 20 is a flow diagram of an example procedure 2000 to predict and report whether a patient is likely to stop a prescribed therapy or reduce treatments to a prescribed therapy, according to an example embodiment of the present disclosure. Although the procedure 2000 is described with reference to the flow diagram illustrated in FIG. 20, it should be appreciated that many other methods of performing the steps associated with the procedure 2000 may be used. For example, the order of many of the blocks may be changed, certain blocks may be combined with other blocks, and many of the blocks described may be optional. In an embodiment, the step of determining recommendations and/or guidelines may be omitted. The actions described in the procedure 2000 are specified by one or more instructions and may be performed among multiple devices including, for example, the clinician server 304 and/or the clinician database 306.

The example procedure 2000 begins in FIG. 20 when the clinician server 304 receives training treatment data 1602 for a patient group (block 2002). The clinician server 304 may identify and select patients that have similar prescribed therapies or programs, patients that have been prescribed a same type of dialysis therapy (e.g., a HD or PD therapy), patients that have received a therapy from a same type of medical fluid delivery machine, and/or patients that have received a prescribed therapy and/or program within the past year. In some instances, the training data includes treatment data for all patients under analysis by the clinician server 304 for the past 6 to 18 months.

The example clinician server 304 uses the training treatment data 1602 to create one or more patient predictive models or engines including, for example, a LightGBM model and/or a neural network model (block 2004). The clinician server 304 then accesses the treatment data 402 and/or the patient data 412 for a patient under analysis (block 2006). The clinician server 304 applies the treatment data 402 and/or the patient data 412 to the one or more patient predictive models to determine a concern score 1604 (block 2008). As discussed above, the concern score includes a probability that the patient will prematurely end (and/or significantly reduce) their prescribed treatments. The clinician server 304 may also determine a top number of critical parameters or attributes that primarily contributed to the concern score (block 2010). This may include at least one critical parameter or as many as ten critical parameters.

The example clinician server 304 may compare the concern score to a threshold (block 2012). If a threshold is exceeded, the clinician server 304 generates an alert, which is transmitted to an application 320 on a clinician device 152 via a document or message 1606 (block 2014). The alert is indicative that a clinician-specified or default threshold has been exceeded for a concern score, and alerts the clinician that the patient is at risk for ending or reducing their treatments. The example clinician server 304 also stores the concern score and critical parameters 1604 (and/or all parameters that contributed to the calculation of the concern score) to one or more patient records in the clinician database 306 of FIG. 16 (block 2016). The clinician server 304 or the web portal 150 may later retrieve the stored data 1604 upon request for rendering and display on an application 320 at a personal mobile communication device 122 or a clinician device 152.

The clinician server 304, in some embodiments, may determine if a recommendation or guideline is to be provided based, for example, on whether an alert was generated or based on the concern score 1604. If a recommendation is to be generated, the clinician server 304 determines the appropriate or corresponding recommendation, and creates a recommendation document 430 for transmission and display via the application 320 (block 2018). The example procedure 2000 of FIG. 20 may then end. The procedure 200 begins again when additional treatment data is received.

### IV. CONCLUSION

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A system for managing patient adherence to a prescribed therapy or program that is administered by an automated peritoneal dialysis (“APD”) machine, the system comprising:
a memory device storing
a record of the prescribed therapy or program for a patient including a schedule of days a treatment is to be provided, a prescribed treatment duration, and an estimated prescribed treatment dwell time, and
treatment data generated by the APD machine, the treatment data being indicative of a treatment duration, a fluid dwell time, and date for each dialysis treatment performed by the APD machine according to the prescribed treatment or program;
an interface device communicatively coupled to the APD machine via a network, the interface configured to receive the treatment data from the APD machine; and
an analytics processor communicatively coupled to the interface device and the memory device, the analytics processor configured to
store the treatment data received by the interface device to the memory device,
determine a lost treatment time parameter value as a difference or ratio between the prescribed treatment duration and the treatment durations of the dialysis treatments,
determine a lost dwell time parameter value as a difference or ratio between the estimated prescribed treatment dwell time and the fluid dwell time of the dialysis treatments,
determine a completed treatment days parameter value as a difference or ratio between the schedule of days the treatment is to be provided and the date of the dialysis treatments, and
cause the lost treatment time parameter value, the lost dwell time parameter value, and the completed treatment days parameter value to be displayed within a user interface on a clinician device.

2. The system of claim 1, wherein the memory device includes a data structure that relates medical fluid delivery recommendations to at least one of a range of lost treatment time parameter values, a range of lost dwell time parameter values, or a range of completed treatment days parameter values.

3. The system of claim 2, further comprising a guidance processor communicatively coupled to the memory device and configured to:
compare at least one of the lost treatment time parameter value, the lost dwell time parameter value, or the completed treatment days parameter value for the patient to the respective at least one of the range of lost treatment time parameter values, the range of lost dwell time parameter values, or the range of completed treatment days parameter values;
select at least one recommendation based on the comparison; and
cause the at least one recommendation to be displayed within the user interface on the clinician device.

4. The system of claim 1, wherein the analytics processor is configured to:
store the lost treatment time parameter value, the lost dwell time parameter value, and the completed treatment days parameter value to the memory device in association with previous lost treatment time parameter values, previous lost dwell time parameter values, and previous completed treatment days parameter values from previous treatments of the patient;
create a first graph using the current and previous lost treatment time parameter values to show actual treatment times for the patient compared to the prescribed treatment duration of the treatments; and
cause the first graph to be displayed in a first user interface of the clinician device.

5. The system of claim 4, wherein the analytics processor is configured to:
create a second graph using the current and previous lost dwell time parameter values to show actual dwell times for the patient compared to the estimated prescribed treatment dwell time; and
cause the second graph to be displayed in a second user interface of the clinician device.

6. The system of claim 1, wherein the record of the prescribed therapy or program for the patient includes at least one of a fill threshold or a drain threshold, and the treatment data includes at least one of a fluid fill time or a fluid drain time for the treatment, and
wherein the analytics processor is configured to generate an alert if the at least one of the fluid fill time, a weekly average of fluid fill times, the fluid drain time, or a weekly average of fluid drain times exceeds the respective at least one of the fill threshold or the drain threshold.

7. The system of claim 6, wherein the alert is indicative of an issue with a catheter of the patient.

8. The system of claim 6, wherein the record of the prescribed therapy or program for the patient includes at least one of a fill threshold or a drain threshold, and the treatment data includes at least one of a fluid fill time or a fluid drain time for the treatment, and
wherein the analytics processor is configured to generate an alert if the at least one of the fluid fill time or the fluid drain time exceeds the respective at least one of the fill threshold or the drain threshold.

9. The system of claim 6, wherein the fluid fill time includes an average fluid fill time for cycles of the treatment and the fluid drain time includes an average fluid drain time for cycles of the treatment.

10. A system for predicting patient stoppage of a prescribed treatment that is administered by an automated peritoneal dialysis (“APD”) machine, the system comprising:
a memory device storing
a training data set including treatment data and patient data for a group of patients, the training data also including an indication as to whether the patients stopped treatments of a prescribed therapy or program,
at least one patient predictive model that is formed using the training data set, the at least one patient predictive model including inputs of at least (i) counts or frequency of alerts generated by an APD machine, (ii) information related to peritoneal dialysis cycles, (iii) patient blood pressure values, and (iv) patient weight values, and
patient data and previous treatment data for a target patient that is undergoing a prescribed therapy or program;
an interface device communicatively coupled to the APD machine via a network, the interface configured to receive the treatment data from the APD machine for a target patient; and
a predictive processor communicatively coupled to the interface device and the memory device, the predictive processor being configured to:
store the treatment data received by the interface device to the memory device,
determine a concern score for the target patient by applying the patient data, the treatment data, and the previous treatment data of the target patent to the at least one patient predictive model, and
cause the concern score to be displayed within a user interface on a clinician device.

11. The system of claim 10, wherein the concern score is indicative of a probability that the target patient will at least one of end treatments or reduce a frequency of treatments of the prescribed therapy or program.

12. The system of claim 10, wherein the predictive processor is configured to:
identify most significant concern parameters that contributed to the concern score; and
cause an indication of the most significant concern parameters to be displayed within the user interface on the clinician device.

13. The system of claim 10, wherein the memory device includes a data structure that relates medical fluid delivery recommendations to a range of concern scores.

14. The system of claim 13, further comprising a guidance processor communicatively coupled to the memory device and configured to:
compare the concern score of the target patient to the range of concern scores;
select at least one recommendation based on the comparison; and
cause the at least one recommendation to be displayed within the user interface on the clinician device.

15. A method for managing patient adherence to a prescribed therapy or program that is administered by a dialysis machine, the method comprising:
receiving, in an interface device, treatment data from the dialysis machine, the treatment data indicative of a treatment duration, a fluid dwell time, and date for dialysis treatments performed by the dialysis machine according to a prescribed treatment or program;
determining, via an analytics processor communicatively coupled to the interface device, dialysis treatment parameter values by comparing the treatment data to a record of the prescribed therapy or program, the record including a schedule of days a treatment is to be provided, a prescribed treatment duration, and an estimated prescribed treatment dwell time, the dialysis treatment parameter values including at least two of
a lost treatment time parameter value as a difference or ratio between the prescribed treatment duration and the treatment durations of the dialysis treatments,
a lost dwell time parameter value as a difference or ratio between the estimated prescribed treatment dwell time and the fluid dwell time of the dialysis treatments, or
a completed treatment days parameter value as a difference or ratio between the schedule of days the treatment is to be provided and the date of the dialysis treatments,
causing, via the analytics processor, the at least two of the lost treatment time parameter value, the lost dwell time parameter value, or the completed treatment days parameter value to be displayed within a user interface on a clinician device.

16. The method of claim 15, further comprising storing, via the analytics processor to a memory device, the received treatment data and the record.

17. The method of claim 15, wherein the dialysis machine includes an automated peritoneal dialysis (“APD”) machine or a hemodialysis machine.

18. The method of claim 15, wherein the record of the prescribed therapy or program for the patient includes at least one of a fill threshold or a drain threshold, and the treatment data includes at least one of a fluid fill time or a fluid drain time for the treatment.

19. The method of claim 18, further comprising at least one of:
generating, via the analytics processor, an alert if the at least one of the fluid fill time, a weekly average of fluid fill times, the fluid drain time, or a weekly average of fluid drain times exceeds the respective at least one of the fill threshold or the drain threshold; or
generating, via the analytics processor, an alert if the at least one of the fluid fill time or the fluid drain time exceeds the respective at least one of the fill threshold or the drain threshold.

20. The method of claim 19, wherein the alert is indicative of an issue with a catheter of the patient.

21. The method of claim 15, further comprising:
accessing, via the analytics processor, a data structure that relates medical fluid delivery recommendations to at least one of a range of lost treatment time parameter values, a range of lost dwell time parameter values, or a range of completed treatment days parameter values;
comparing, via the analytics processor, at least one of the lost treatment time parameter value, the lost dwell time parameter value, or the completed treatment days parameter value for the patient to the respective at least one of the range of lost treatment time parameter values, the range of lost dwell time parameter values, or the range of completed treatment days parameter values;
selecting, via the analytics processor, at least one recommendation based on the comparison; and
causing, via the analytics processor, the at least one recommendation to be displayed within the user interface on the clinician device.
